(19) United States
(12) Patent Application Publication
(10) Pub. No.: US 2022/0213427 A1
(43) Pub. Date: Jul. 7, 2022

(54) BIOREACTOR DEVICE AND METHODS

(71) Applicant: ARBOREA LTD, London (GB)

(72) Inventor: Julian Paul MELCHIORRI, London (GB)

(73) Assignee: ARBOREA LTD, London (GB)

(21) Appl. No.: 17/608,300

(22) PCT Filed: May 4, 2020
(86) PCT No.: PCT/IB2020/054213

§ 371 Date: Nov. 2, 2021

(30) Foreign Application Priority Data
May 3, 2019 (GB) 1906298.3

(51) Int. Cl. C12M 1/00; B01D 69/04; B01D 53/22; B01D 69/02; B01D 71/70; C12M 1/12; C12M 1/04; C12M 1/34

(52) U.S. Cl. CPC C12M 29/04; B01D 69/04; B01D 53/226; B01D 69/02; B01D 2325/04; C12M 23/58; C12M 23/06; C12M 23/24; C12M 41/12; B01D 71/70

(57) ABSTRACT

An apparatus is provided for the production of biomass or a bioproduct, the apparatus comprising: at least one elongate bioreactor, the bioreactor comprised of at least one outer membrane layer that defines a substantially tubular compartment that is capable of being filled with a liquid or gel, wherein the membrane layer is comprised of a material that is permeable to gas transfer across the membrane layer. A chamber is provided comprising walls that define and enclose a gaseous atmosphere within. At least a part of the bioreactor is located inside the chamber. A control system controls the composition of the atmosphere within the chamber and gas transfer occurs across the membrane layer of the bioreactor between the tubular compartment and the atmosphere comprised within the chamber. Methods of using the apparatus in order to manufacture biomass are also provided.

### FIELD

The invention is in the field of biomass production, particularly via the use of microbial or cellular bioreactors.

### BACKGROUND

Organisms undertaking aerobic respiration consume oxygen and produce carbon dioxide and heat. In a high density, high growth environment, it is necessary to provide oxygen to the microorganisms, as well as to remove CO₂, metabolic waste and excess heat, in order to encourage maximum growth rates.

Chemoheterotrophic microorganisms (which cannot fix carbon to make organic compounds and must consume organic matter from external sources) such as yeast have been grown in the same way for centuries, that is, in large tanks and more recently in batch fermenter tanks. However, fermenter tanks are primarily designed to allow fermentation, being a specific metabolic process which works in the absence of oxygen, with the intended product for the market usually being the fermented by-product (for example, alcohol produced by the fermentation of yeast).

When a market need for the entire biomass of a microorganism, or the products contained within their cells (that is, beyond only its fermented byproducts) arose in the 20^{th} century, existing fermenter tanks were modified, with aerators installed on the bottom of the tanks in order to deliver oxygen or oxygen-containing gas. This enabled the contained microorganisms to perform cellular aerobic respiration within the fermenter tank. Furthermore, modifications were sometimes made with this aeration in mind, for example making the aerating-fermenter tanks tall and thin, to increase the retention time of oxygen bubbles while they travel vertically to the top of the liquid growth medium, or broth.

Because of such adaptations to enable aerobic respiration within tanks formerly intended for fermentation, the conventional design results in inefficient, complex and costly production of biomass or cellular products, for at least the following reasons:
High energy costs, equipment requirements and associated complexity due to the need to sterilise the inlet air for aeration.
High energy costs, equipment requirements and complexity due to the need to compress and deliver oxygen (usually in the form of air).
High energy costs, equipment requirements and complexity due to the need to mix the liquid media, especially at high cell densities (i.e. stirrers and stirring mechanisms).
Capital costs for air compressors, filters and other equipment needs.
Foam formation resulting from aeration, increasing costs for anti-foaming agents, and potential decreasing production quality due to biomass loss in the foam produced.
Difficulties in controlling temperature within tanks; as these are solid and sealed, they generally require cooling water jackets, meaning higher capital costs and energy costs to chill the water.
Contamination risk due to the numerous air-sparging nozzles, valves, sensor ports, paddles, inlets, agitator housings and so on, which provide high risk sites for contamination and are difficult to clean and sterilise.
Risk of the introduction of contaminants such as fungal spores and bacteria despite filtration of input air, due to the need for continuous aeration. Estimations by industry experts suggest that as much as 30% of the total biomass in industrial fermenters may be affected by contamination, decreasing quality and end product yield.
Expensive cleaning costs, due to easy formation of biofilm on stainless steel and necessary aeration-associated features, which is hard to remove only with steam thereby in some cases requiring increased labour costs.
The necessity in most cases to operate in batch procedures, leading to a decrease in yearly yield due to downtime required for cleaning and subsequent re-growth to desired density.

The transfer of gas into bioreactors is usually achieved through the use of aeration technologies, such as by compressing CO₂, O₂, or air, and delivering the compressed gas into the liquid media through nozzles, or by bubbling or sparging the gas into the liquid media (see for example US2015/0230420, WO2015/116963). These techniques can be used to add a desired gas, or can also work to remove excess gas which is not wanted (see for example US2015/0093924).

Techniques of this kind can be disadvantageously inefficient in both energy requirements and infrastructure cost. When a soluble gas is bubbled through a liquid, only a small proportion of the gas will be successfully dissolved; consequently the remaining gas is wasted, leading to a waste of energy and inefficient gas uptake. Gas removal by this technique is limited by the gas which can be trapped in the bubbles produced, which provide only a limited surface area for effective gas exchange.

For example, Aerobic Stirred Fermenters are commonly used which have a high height to diameter ratio (around 3 to 1), and use gas sparged at the bottom of the tank to deliver oxygen and remove carbon dioxide, and also requires the use of active stirring and heat-exchange cooling methods.

Similarly, Air-lift Fermenters of the common internal loop type have a very high height to diameter ratio (around 5 to 1), with mixing provided by the movement of liquid and gas up a central cylinder, with the liquids returning in down-flow in the surrounding annular spaces to deliver oxygen, to remove carbon dioxide, and to allow heat-exchanging cooling methods as the mass of the down-flowing liquids hinders transfer from the central core. Both of these approaches have high operational and capital costs, and have considerable contamination risk from gas inlets (despite sterilisation of the input gas).

WO 2005/100536 A1 describes an incubator and an incubating method capable of incubating a plurality of kinds of cell preferring different gas concentrations simultaneously without requiring a plurality of incubators. The incubator is not suitable for containing a continuous flow circuit of medium but looks like a static incubator that moves cells within a fixed volume of media by agitation or rotation. No system to automatically harvest biomass is described, nor any particular reasoned suitability for cell or microorganism type. No detail on the properties of the materials needed for the apparatus is included, for example in terms of gas permeability, gas pressure, or structural arrangements for improved gas transfer is described.

The present invention addresses the problems that exist in the prior art, not least the production of valuable products from biomass and cellular material, and provides simple and cost-effective solutions to the problems posed by culturing large volumes of organisms, providing them with sufficient oxygen and/or other required gases, and producing biomass. These and other uses, features and advantages of the invention should be apparent to those skilled in the art from the teachings provided herein.

### SUMMARY

In one aspect, there is provided an apparatus for the production of biomass or a bioproduct, the apparatus comprising at least one elongate bioreactor, the bioreactor comprised of at least one outer membrane layer that defines a substantially tubular compartment that is capable of being filled with a liquid or gel, wherein the membrane layer is comprised of a material that is permeable to gas transfer across the membrane layer. The apparatus also comprises a chamber comprising walls that define and enclose a gaseous atmosphere within, wherein at least a part of the bioreactor is located inside the chamber. Also comprised is a control system which controls the composition of the atmosphere within the chamber. In use, gas transfer occurs across the membrane layer of the bioreactor between the tubular compartment and the atmosphere comprised within the chamber.

The walls of the chamber may be substantially rigid or flexible. The chamber may be in the form of a tank, a vessel, a barrel, a tent, a warehouse, an inflated structure, or a room. The atmosphere within the chamber may be elevated to a pressure greater than or less than atmospheric pressure. Substantially all of the bioreactor may be located inside the chamber. The chamber may further comprise a sterilisation system, gas circulatory apparatus, and/or a source of illumination, optionally wherein the source of illumination emits visible and/or UV light. Such a source of illumination may be sporadic or intermittent. In some embodiments, at least one or a part of one wall of the chamber permits the transmission therethrough of visible light into the interior of the chamber.

In some embodiments, the control system is configured to alter the atmospheric composition of the chamber by one or more of the introduction of O₂, for example in the form of atmospheric air, suitably prefiltered air); the depletion of CO₂ concentration; and the introduction of steam.

In some embodiments, the chamber comprises an assembly for supporting the at least one elongate bioreactor within. The assembly may comprise a plurality of shelves arranged in either a horizontal or vertical parallel or anti-parallel array. The shelves may comprise a cradle configured to support the at least one elongate bioreactor. The cradle may substantially enclose all or a part of the elongate bioreactor. The cradle may be comprised of a mesh and/or a perforated sheet material, such that atmospheric circulation may be permitted via the perforations of the sheet material. The cradle may be planar or curved. in some embodiments the cradle may be a solid sheet without holes or perforations and made of any suitable material capable of affording support to the bioreactor (for example metal, aluminium, steel, and/or polymer/plastic). In one embodiment the base of the chamber is integrated into the cradle structure in order to support the elongate bioreactor, in which case the base of the chamber is suitably comprised of a solid formed or moulded sheet of any suitable material as shown in FIG. 15.

In some embodiments, the elongate bioreactor is comprised of one or more hose sections, wherein each hose section is comprised of a gas permeable polymer membrane. In some embodiments, the gas permeable polymer membrane comprises a material selected from: silicones, polysiloxanes, polydimethylsiloxanes (PDMS), fluorosilicone, organosilicones, Vinyl Methyl Siloxane (VMQ), Phenyl vinyl methyl siloxane (PVMQ), silicon-oxide polymers, sulfonated polyetheretherketone (SPEEK), poly(ethylene oxide), poly(butylene terephthalate), or poly(ethylene oxide), poly(butylene terephthalate) block copolymers (PEO-PBT), cellulose (including plant cellulose and bacterial cellulose), cellulose acetate (celluloid), nitrocellulose, and cellulose esters. The membrane may be an elastomer. In some embodiments, the membrane has an oxygen permeability of at least 350, at least 400, at least 450, at least 550, at least 650, at least 750, suitably at least 820 Barrers. The membrane may have a carbon dioxide permeability of at least 2000, at least 2500, at least 2600, at least 2700, at least 2800, at least 2900, at least 3000, at least 3100, at least 3200, at least 3300, at least 3400, at least 3500, at least 3600, at least 3700, at least 3800, suitably at least 3820 Barrers. The membrane may have a water vapour permeability of not less than about 5000 Barrer, suitably not less than about 10000 Barrer, about 15000 Barrer, about 20000 Barrer, 25000 Barrer, about 30000 Barrer, about 35000 Barrer, about 40000, about 60000 and typically at least about 80000 Barrer.

The membrane may have a thickness of at least 10 μm and at most 1 mm, suitably at least 20 μm and at most 500 μm, optionally at least 20 μm and at most 200 μm.

In some embodiments, the one or more hose sections are joined by one or more connectors that facilitate fluid communication between the one or more hose sections. The one or more hose sections may be formed with variable membrane thickness such that a portion of the membrane proximate to the one or more connectors is thicker than a portion of the membrane distant to the one or more connectors. The apparatus may comprise a plurality of hose sections joined by one or more connectors that facilitate fluid communication between the plurality of hose sections, and wherein the thickness of the membrane between hose sections is dependent upon the vertical positioning of the of the hose section within the chamber. The connectors used in the apparatus may comprise valves configured to selectively prevent or allow passage of liquid media through the connector.

The bioreactors of the invention may be in fluid communication with an auxiliary system, The one or more bioreactor may comprise a cellular growth medium. The one or more bioreactor may comprise a microbial or algal organism selected from a: chemotroph and a mixotroph. The bioreactor may comprise an organism selected from one or more of Cyanobacteria, Protobacteria, Spirochaetes, Gram Positive bacteria, green filamentous bacteria such as Chloroflexia, Planctomycetes, Bacteroides cytophaga, Thermotoga, Aquifex, halophiles, Methanosarcina, Methanobacterium, Methanococcus, Thermococcus celer, Thermoproteus, Pyrodictium, Entamoebae, slime moulds such as Mycetozoa, Ciliates, Trichomonads, Microsporidia, Diplomonads, Excavata, Amoebozoa, Choanoflagellates, Rhizaria, Foraminifera, Radiolaria, Diatoms, Stramenopiles, brown algae, red algae, green algae, snow algae, Haptophyta, Cryptophyta, Alveolata, Glaucophytes, phytoplankton, plankton, Percolozoa, Rotifera, and cells or whole organisms from animals, fungi, bacteria or plants.

In some embodiments, the bioreactor comprises a eukaryotic cell culture; suitably an animal or plant cell culture; optionally a mammalian cell culture. An animal cell culture may comprise cells selected from one or more of myocyte cells, adipocyte cells, epithelial cells, myoblasts, satellite cells, side population cells, muscle derived stem cells, mesenchymal stem cells, myogenic cells, myogenic pericytes, or mesoangioblasts. The bioreactor may comprise a human cell culture.

In another aspect, there is provided a method for manufacturing biomass, the method comprising providing an apparatus as described above. In particular, the apparatus comprises at least one elongate bioreactor, the bioreactor comprised of at least one outer membrane layer that defines a substantially tubular compartment that is capable of being filled with a liquid or gel, wherein the membrane layer is comprised of a material that is permeable to gas transfer across the membrane layer. The apparatus further comprises a chamber comprising walls that define and enclose a gaseous atmosphere within wherein at least a part of the at least one bioreactor is located inside the chamber and a control system which controls the composition of the atmosphere within the chamber. The at least one elongate bioreactor comprises a liquid cellular growth medium and a microbial or algal organism selected from a chemotroph and a mixotroph, and/or a eukaryotic cell culture. The method comprises culturing the organisms or cell cultures within the one or more bioreactors of the apparatus, and separating at least a part of the biomass present within the liquid media.

### DRAWINGS

The invention is further illustrated by reference to the accompanying drawings in which:

FIGS. 1A and 1B are diagrams showing cross-sections of devices according to an embodiment of the invention having a linear bioreactor with an inlet and an outlet located on opposite sides, disposed within a gas-filled chamber also provided with an inlet and outlet.

FIG. 2 shows a cross-section of an arrangement according to another embodiment of the invention wherein two bioreactors are directly connected in series.

FIGS. 3a and 3b show cross sections of an arrangement according to another embodiment of the invention wherein two bioreactors are directly connected in series, wherein each bioreactor is contained within a chamber.

FIG. 4 shows a cross section of an arrangement according to another embodiment of the invention where five pairs of bioreactors are connected in series.

FIG. 5 shows a cross section of an arrangement according to another embodiment of the invention where five pairs of bioreactors are connected in parallel.

FIGS. 6a to 6d show arrangements of arrays of bioreactors which may be used in some embodiments of the invention.

FIGS. 7a and 7b show planar sections A and B through representations of the device according to some embodiments of the invention.

FIGS. 8a and 8b show additional features which may be comprised within connectors or conduits of systems according to some embodiments of the invention.

FIG. 9 shows a suitable system of one embodiment of the invention, comprising any embodiment of one or more bioreactors and an associated auxiliary system.

FIG. 10 shows a cross section of a support member for use with a device according to embodiments of the invention.

FIG. 11 shows a cross-section of a device according to an embodiment of the invention comprising bioreactors supported on a support member.

FIG. 12 shows a perspective view of support members for use with a device according to embodiments of the invention.

FIG. 13 shows a cross-section of a device according to an embodiment of the invention comprising a convex curved upper chamber wall, to encourage runoff under gravity of water, snow, sand and other substances that might deposit on an interior or exterior surface.

FIGS. 14a to 14d show views of bioreactors supported on support structures and/or bioreactor support structures in accordance with some embodiments of the invention. FIGS. 14a and b show a cross-section of an array of bioreactors supported on shelf-like support structures. FIG. 14c shows a perspective view of an example of a bioreactor being supported, contained, and reinforced with a surrounding mesh. FIG. 14d shows a side view of an array of bioreactors supported on shelf-like support structures.

FIG. 15a shows a cross-section of an array of bioreactors supported on a flat support structure that also defines the base of the chamber, and a convex curved upper chamber wall to increase its structural strength and to encourage runoff under gravity of substances that might deposit on an interior or exterior surface, in accordance with some embodiments of the invention.

FIG. 15b shows a cross-section of an array of bioreactors supported on flat support structures that define the base of multiple chambers, and integrated illumination devices, in accordance with some embodiments of the invention. The integrated illumination may be used to sustain the growth of phototrophic and/or mixotrophic organisms.

FIG. 15c shows a cross-section of an array of bioreactors supported on planar support structures, in accordance with some embodiments of the invention.

FIGS. 16a to 16c show a cross-section of a bioreactor being formed by a single membrane layer folded to form an elongate seam and joined on itself. FIG. 16a shows how a single membrane layer may be folded before the two edges are bonded to define a bioreactor within. FIG. 16b shows a bioreactor formed by a single membrane layer folded and glued to itself. FIG. 16c shows a bioreactor formed by a single membrane layer folded and bonded to itself and where the bonded section also provides additional structural reinforcement on the lower side of the bioreactor in contact with the planar support structure.

FIG. 17a shows a perspective view of an example bioreactor with end reinforcements.

FIG. 17b shows a perspective view of an example bioreactor with both end reinforcements and a continuous lower reinforcement structure.

FIG. 18 shows a suitable system of one embodiment of the invention used for the experiments described in Example 1, comprising a bioreactor system and an associated auxiliary system.

FIG. 19 shows a suitable system of one embodiment of the invention used for the experiments described in Example 2, comprising a bioreactor system and an associated auxiliary system that includes a source of illumination (either natural or artificial).

FIG. 20 shows the results of the Example 1 in the form of a graph of the optical density in the liquid media for both experimental runs (Run A and Run B).

FIG. 21 shows the results of the Example 1 in the form of a graph of the temperature in the liquid media.

FIG. 22 shows the results of the Example 2 in the form of a graph of the optical density in the liquid media.

FIG. 23 shows the results of the Example 2 in the form of a graph of the temperature in the liquid media.

### DETAILED DESCRIPTION

All references cited herein are incorporated by reference in their entirety. Unless otherwise defined, all technical and scientific terms used herein have the same meaning as commonly understood by one of ordinary skill in the art to which this invention belongs.

The present inventor has developed a gas permeable bioreactor device suitable for generating biomass, comprised within a chamber. Advantageously, the atmosphere within the chamber can be controlled in order to supply the bioreactor device with a gaseous feed of specified composition as well as removing effluent gas. Embodiments of the invention permit the specified device to comprise an atmosphere that is optimised in order to improve or maximise organism survival, organism growth rate and/or biomass production within the bioreactor. Alternative embodiments of the invention permit for the specified device to comprise an atmosphere that controls growth of or modulates biomolecule synthesis by a microorganism comprised within the bioreactor. These and other embodiments of the invention are described in more detail below.

Prior to further setting forth the invention, a number of definitions are provided that will assist in the understanding of the invention.

As used herein, the term “comprising” means any of the recited elements are necessarily included and other elements may optionally be included as well. “Consisting essentially of” means any recited elements are necessarily included, elements that would materially affect the basic and novel characteristics of the listed elements are excluded, and other elements may optionally be included. “Consisting of” means that all elements other than those listed are excluded. Embodiments defined by each of these terms are within the scope of this invention.

As used herein, the terms ‘autotroph’, ‘autotrophy’ or ‘autotrophic’ refers to organisms and processes which can produce complex organic molecules from inorganic chemicals in its environment. In particular, this means the fixation of carbon, typically carbon dioxide, into organic compounds. The energy required for this may come from light or from chemical reactions. Photosynthesis is an example of an (photo)autotrophic process. Chemoautotrophic organisms, defined below, use energy obtained from chemical reactions to fix inorganic carbon (for example from carbon dioxide) into organic compounds.

As used herein, the terms ‘heterotroph’, ‘heterotrophy’ or ‘heterotrophic’ refers to organisms and processes which are unable to fix carbon to form organic compounds, that is, they consume organic matter from their surroundings and convert them into organic molecules for their own use.

As the skilled person will be aware, the term “photosynthesis” refers to a biochemical process that takes place in green plants and other photosynthetic organisms, including photosynthetic microorganisms including algae and cyanobacteria. The process of photosynthesis utilises electromagnetic waves (light) by photon capture as an energy source to convert carbon dioxide and water to metabolites and oxygen. As used herein, the term “photosynthetic microorganism” refers to any microorganism that is capable of photosynthesis. As used herein, the related terms “photosynthetic” and “photosynthesising” are synonymous with to “photosynthetic” and the two terms can be used interchangeably herein.

As used herein, the terms ‘phototroph’, ‘phototrophy’ or ‘phototrophic’ refer to any organism or process which can capture energy from light for any purpose, in particular organisms and processes which produce energy and/or produce organic compounds using energy from electromagnetic waves (light) by photon capture. As mentioned above, the production of organic compounds by fixation of inorganic carbon using energy from light is known as photosynthesis. A “photoautotroph” as the term is used herein is another term for an organism that can produce organic compounds from carbon dioxide with energy from light. As described below, photosynthetic organisms and photoautotrophs are not restricted to using photosynthesis alone, and many organisms may use or be capable of photosynthesis. In addition, some organisms use light to provide cellular energy (such as in the form of ATP), but are not necessarily capable of fixing carbon to produce organic compounds. A “photoheterotroph”, as the term is used herein, refers to an organism which can generate cellular energy from light, but cannot fix (sufficient) inorganic carbon to supply its needs.

As used herein, the terms ‘chemotroph’, ‘chemotrophy’ or ‘chemotrophic’ refer to organisms and processes that obtain energy by the oxidation of electron donors in their environments. These molecules can be organic (chemo-organotrophs) or inorganic (chemolithotrophs). Chemotrophs can be either autotrophic or heterotrophic. For example, an organism which consumes organic carbon compounds from its environment and oxidises these compounds to produce ATP is a chemotroph. ‘Chemoheterotrophs’, a term which includes most animals and fungi, refers to organisms which consume organic compounds from external sources and use them to form their own organic compounds, rather than fixing carbon directly to make organic compounds. ‘Chemoautotrophs’ are organisms which can use energy obtained from chemical reactions to fix inorganic carbon (for example from carbon dioxide) into organic compounds. Examples of such chemical energy sources include hydrogen sulfide, elemental sulfur, ferrous iron, molecular hydrogen, and ammonia. Many chemoautotrophs are extremophiles, bacteria or archaea that live in hostile environments, and are the primary producers in such ecosystems. Chemoautotrophs generally fall into several groups: methanogens, halophiles, sulfur oxidisers and reducers, nitrifiers, anammox bacteria, thermoacidophiles, Manganese oxidisers, Iron-oxidisers, and hydrogen oxidisers. For example, hydrogen oxidising bacteria can oxidise hydrogen as a source of energy, using oxygen as the final electron acceptor. Similarly, methanogens are microorganisms that produce methane as a metabolic byproduct, in conditions of low oxygen, and some methanogens use hydrogen to reduce carbon dioxide into methane and water.

As used herein, the terms ‘mixotroph’, ‘mixotrophy’ or ‘mixotrophic’ refer to organisms and processes which can use more than one source of energy and/or organic compounds. Most often, this refers to organisms which can use a mixture of light and chemical inputs to acquire or produce energy and/or organic compounds. Mixotrophic organisms exist on a spectrum between full obligate chemoheterotrophy and full obligate photoautotrophy. Using such a mixture of sources may be obligate, where an organism must use the mixture of sources to survive, or facultative, where the organism uses one source preferentially and the other under particular circumstances, for example using chemical sources of energy where light is limiting. Therefore, a ‘mixotrophic organism’ is both a phototroph and a chemotroph, and may be a photoautotroph, a chemoautotroph, a photoheterotroph, or a chemoheterotroph.

The skilled person will also be aware that references to the concentration or percentage of CO₂ (carbon dioxide) in liquid refers to the dissolved inorganic carbon (DIC) of the solution, that is, the concentration of dissolved CO₂ as well as related inorganic species H₂CO₃ (carbonic acid), HCO₃⁻ (bicarbonate) and CO₃²⁻ (carbonate). Similarly, references herein to “gas concentration” and the like are intended to include any and all ionic species or chemical compounds which form from gases in a liquid or aqueous context, for example ammonium ions (NH₄⁺) as a result of ammonia gas or sulphuric acid (H₂SO₄) as a result of sulphur oxides.

As used herein, the term “translucent” has its ordinary meaning in the art, and refers to a light-pervious material that allows light to pass through, resulting in the random internal scattering of light rays. The term is synonymous with “semi-transparent”.

As used herein, the term “transparent” has its ordinary meaning in the art, and refers to a material that allows visible light to pass through it, such that objects can be clearly seen on the other side of the material, in other words it can be described as “optically clear”. All membrane and non-membrane materials, chamber walls, additional components, control structures, coatings and other materials described herein can be substantially translucent or substantially transparent.

As used herein, the term “permeable” or “gas permeable” means a material that allows gases, in particular some or all of oxygen (O₂), carbon dioxide (CO₂), nitrogen (N₂), water vapour (H₂O) and, optionally, methane (CH₄) and/or sulphur dioxide (SO₂) to be transferred from one side of the material to the other, in either or both directions. As used herein, the related terms “breathable” and “semipermeable” are synonymous with “permeable” and the two terms can be used interchangeably herein. Typically, the material is in the form of a sheet, film or membrane. The permeation is directly related to the concentration gradient of the permeant (such as gas), a material's intrinsic permeability, and the diffusivity of the permeant species in the membrane material.

Permeability of a gas through a specific material is measured herein in Barrers. The Barrer measures the rate of a gas flow passing through an area of material with a thickness, driven by a given pressure. Barrer is defined as:

$$1 Barrer = 1 {0}^{- 10} \frac{{cm}_{STP}^{3} \cdot cm}{{cm}^{2} \cdot s \cdot cm Hg}$$

It will be appreciated that the Barrer is the most common measurement of gas permeability in current usage, particularly in relation to gas-permeable membranes, however permeability may also be defined by other units, examples of which include kmol·m·m−2·s−1·kPa−1, m3·m·m−2·s−1·kPa−1, or kg·m·m−2·s−1·kPa−1. ISO 15105-1 specifies two methods for determining the gas transmission rate of single-layer plastic film or sheet and multi-layer structures under a differential pressure. One method uses a pressure sensor, the other a gas chromatograph, to measure the amount of gas which permeates through a test specimen. Other equivalent measurements of gas-permeability are known to the skilled person and would be readily equivalent to Barrer measurements described herein.

As used herein, the term “biomass” refers to any living or dead microorganism, including any part of a microorganism (including metabolites and by-products produced and/or expelled by the microorganism).

As used herein, the term a “device” may be comprised of one “unit”, or may comprise an array or combination of a plurality of “units”.

As used herein, the term ‘chamber’ also refers to a ‘gas chamber’ and the two terms can be used interchangeably herein.

As used herein, the term “fluid” refers to a flowable material, typically a liquid and suitably liquid media, which is comprised within the units, and thus the devices of the invention. “Fluid” may also be used to describe a gas, such as the atmosphere which is comprised within the chambers of the invention.

As used herein, the term “liquid media” has its usual meaning in the art and is a liquid used to grow the organisms and which contains the organisms. The liquid media can comprise one or more of the following: fresh water, salty water, saline, brine, sea water, waste water, sewage, nutrients, phosphates, nitrates, vitamins, minerals, micronutrients, macronutrients, metals, digestate, fertilisers, microorganism growth media, BG11 growth media, PYGV media, and organisms. The liquid media can in particular also comprise carbon sources for the comprised organisms; often these are glucose sources. Suitable carbon sources of this kind can include lignin, cellulose, hemi-cellulose, starch, xylan, polysaccharide, xylose, galactose, sucrose, lactose, glycerol, molasses or glucose, or derivatives thereof. Due to the high density of microorganisms which it is possible to support in devices of the present invention, the term liquid media is intended to encompass a wide range of viscosities, including substantially gel-like or semisolid compositions.

As used herein, terms relating to the orientation of the device of the invention are generally used in their commonly held meanings, but are also intended to vary as appropriate depending on the particular intention or configuration of the invention. Thus, terms such as upper, top and above may refer to directions away from the Earth's gravity. Similarly, terms such as lower, bottom and below refer to directions towards the Earth's gravity.

The present invention uses gas-permeable membrane bioreactors of the general class described for the cultivation of photosynthetic organisms in WO2017/093744 and WO2018/100400, but further adapted to provide application to organisms with a diverse range of trophic capabilities. This approach overcomes several problems seen with existing bioreactor systems because it enables, in part, much less energy intensive gas-transfer control in the liquid media, including on a large scale, and provides greater versatility compared to systems that require devices for controlling aeration and compression of feed gases administered directly to the liquid media. The operational complexity and extra weight associated with compression and aeration techniques is also avoided. Due to the nature of the invention, the natural expansion properties of gas mean that supplied gas can be easily supplied and expand to rapidly change the composition of the entire chamber. This provides a further benefit, as the gas concentration within the chamber can be relatively easily controlled on a large scale, and by extension the gas concentration in the liquid media can be controlled on the same scale.

In cases of high growth rate of cultivated organisms or in other cases where a bioreactor is exposed to sunlight or to any other source of heat (natural or artificial source of heat), large amounts of excess heat may be generated and/or collected in a bioreactor, which can damage or kill the organisms contained within a bioreactor. The membranes of the bioreactors of the invention are in some embodiments permeable to water vapour, and the dissipation of this vapour represents an efficient method of heat shedding from the liquid media, thereby further improving heat control. Further, the large surface area provided by the membranes of the bioreactor which is in contact with the atmosphere within the chamber and the thin wall thickness of the membrane layer of the bioreactor also provides for efficient heat transfer through contact with the surrounding gaseous atmosphere in the chamber. Therefore, the present invention can control the liquid temperature by controlling the temperature of the gaseous atmosphere within the chamber. This particular method enables a constant heat exchange throughout the length of the bioreactor and permits maintenance of a substantially homogeneous liquid media temperature throughout the length of the bioreactor, independently from its length; on the contrary, conventional heat exchanging methods (utilised by standard bioreactors) modify the temperature of the liquid media only in a specific section of the bioreactor system. This is suitable for single vessel bioreactors but can be problematic for bioreactors that are elongate (e.g. based on a tubular liquid circuit as described herein) because and they are not able to maintain an homogeneous liquid media temperature throughout the bioreactor length. This is due to the fact that after the liquid media travels through the heat exchanger and its temperature is modified, its temperature will constantly change during its circulation throughout the bioreactor system. The thickness of the membrane layer of the bioreactor can be suitably modified to increase or decrease the heat transfer rate (i.e. heat transfer coefficient) and the gas transfer rate between the liquid media and the gaseous atmosphere within the chamber.

Another benefit of the present invention is in increasing the robustness and environmental resistance of a bioreactor comprised within an assembly. The walls of the chamber may be configured to provide thermal insulation against external factors such as changing environmental or seasonal conditions. This insulation also decreases the energy necessary for the maintenance of the temperature of liquid media comprised with the bioreactors. Physical protection of the potentially fragile membrane of the bioreactor is also provided against factors such as weather, wind or hail, or animal damage. The provision of an additional barrier also acts to contain spills from the bioreactor into the environment.

Further, the nature of the device of the invention means that processes of cleaning and sterilisation can be carried out effectively and efficiently. According to one embodiment of the invention, the tubular configuration of the membranes which comprise and contain the liquid media allows for the removal of blind endings, corners, edges, seams and other crevices, by enabling a substantially uniform cross-section of the bioreactor. Since such features provide areas where unwanted microorganisms and biofilms can attach, or where debris, spent liquid media or other detritus could accumulate, as well as being difficult to clean effectively, the present invention allows for fast and efficacious cleaning to take place. The absence of necessary gas bubbling or sparging techniques also means that the nozzles, outlets and inlets required for such techniques will not be in contact with the liquid media or organisms, and therefore will not have to be cleaned. Such features can be difficult to clean and are frequently areas of microbial growth or debris collection, and can even be sources of contamination themselves through the introduction of contaminants with the input gas. Therefore, the invention allows for increased sterility and flexibility in process setup and shut down, as cleaning before and after use can be more effective.

### The Bioreactor

According to one embodiment of the invention, the bioreactor of the device is provided that comprises at least one outer layer that is a membrane layer. The membrane layer or layers may be flexible. At least a part of one of the membrane layers, and optionally substantially all of each of the membrane layers, is permeable to transmission of gases across the membrane. As used in this context, the phrase “at least a part” means an area of the layer that is of a sufficient size to allow a gas to pass through the outer layer of the bioreactor. The gas is typically oxygen, carbon dioxide and water vapour, but not limited thereto, and may comprise nitrogen, nitrogen oxides, sulphur oxides, hydrogen and/or methane.

The permeability coefficient of oxygen through the membrane may be not less than about 100 Barrer, suitably not less than about 200 Barrer, about 300 Barrer, about 400 Barrer, about 500 Barrer, about 600 Barrer, about 700 Barrer, about 800 Barrer, about 900 Barrer, about 1000 Barrer, about 1250 Barrer, about 1500 Barrer, and typically not less than about 2000 Barrer.

The permeability coefficient of carbon dioxide through the membrane may be not less than about 100 Barrer, suitably not less than about 200 Barrer, about 400 Barrer, about 600 Barrer, about 800 Barrer, about 1000 Barrer, 1500 Barrer, about 2000 Barrer, about 2500 Barrer, about 3000 Barrer, about 3500 Barrer, about 4000 Barrer, about 4500 Barrer, about 5000 Barrer, about 7500 and typically not less than about 10000 Barrer.

The permeability coefficient of water vapour through the membrane may be not less than about 5000 Barrer, suitably not less than about 10000 Barrer, about 15000 Barrer, about 20000 Barrer, 25000 Barrer, about 30000 Barrer, about 35000 Barrer, about 40000, about 60000 and typically not less than about 80000 Barrer. Water Vapour permeability can also be measured in g/m²/24 h. In these terms, suitable water vapour permeability through the membrane may be around 3200 at a membrane thickness of 20 μm, 1200 at a thickness of 50 μm and 800 at a thickness of 100 μm.

Where the membrane is permeable to methane (CH₄), the permeability coefficient of methane through the membrane may be not less than about 100 Barrer, suitably not less than about 250 Barrer, about 500 Barrer, about 600 Barrer, 700 Barrer, about 800 Barrer, about 900 Barrer, about 1000, about 1500 and typically not less than about 5000 Barrer.

Where the membrane is permeable to sulphur dioxide (SO₂), the permeability coefficient of sulphur dioxide through the membrane may be not less than about 1000 Barrer, suitably not less than about 2500 Barrer, about 5000 Barrer, about 6000 Barrer, about 7000 Barrer, about 8000 Barrer, about 9000 Barrer, about 10000, about 12000, about 14000, and typically not less than about 16000 Barrer. Typically, the permeability of sulphur dioxide is around 12500 Barrer.

Where the membrane is permeable to hydrogen sulphide (H₂S), the permeability coefficient of hydrogen sulphide through the membrane may be not less than about 1000 Barrer, suitably not less than about 2500 Barrer, about 5000 Barrer, about 6000 Barrer, about 7000 Barrer, about 8000 Barrer, about 9000 Barrer, about 10000, and typically not less than about 12000 Barrer. Typically, the permeability of hydrogen sulphide is around 8400 Barrer.

Where the membrane is permeable to molecular hydrogen (H₂), the permeability coefficient of molecular hydrogen through the membrane may be not less than about 100 Barrer, suitably not less than about 250 Barrer, about 500 Barrer, about 600 Barrer, 700 Barrer, about 800 Barrer, about 900 Barrer, about 1000, about 1500 and typically not less than about 2000 Barrer. Typically, the permeability of molecular hydrogen is around 550 Barrer.

Where the membrane is permeable to molecular nitrogen (N₂), the permeability coefficient of molecular hydrogen through the membrane may be not less than about 50 Barrer, suitably not less than about 100 Barrer, about 200 Barrer, about 300 Barrer, 500 Barrer, about 700 Barrer, about 900 Barrer, about 1000, about 1500 and typically not less than about 2000 Barrer. Typically, the permeability of molecular nitrogen is around 200 Barrer.

The bioreactor may be exposed to a source of illumination, whether artificial or natural, from a single direction or from multiple directions. If the bioreactor is positioned such that it receives light primarily from a single direction and one (first) membrane layer is less transparent or less translucent than another (second) membrane layer, the first membrane layer can be on the side of the bioreactor which faces the primary light source. It is contemplated in some cases that the membrane layer may be substantially opaque or impermeable to visible light, and that no light source may be included or intended. Typically, the membrane layer is at least translucent, and is suitably substantially transparent to allow visual inspection of the contents of the bioreactor.

Typically, a membrane layer comprises one or more gas permeable materials. It is important that the gas permeable material is not permeable to liquids, to prevent liquid media within the bioreactor leaking to the outside. The gas permeable material can be porous (including microporous structure gas permeable materials) or non-porous. Gas permeable materials are referred to as porous if the gas particles can migrate through direct movement through a microporous structure. If the gas permeable material is porous, it is important that it is substantially impermeable to liquids. Suitably, the gas permeable material is non-porous, this to avoid also liquid permeation through the gas permeable material and to avoid lower transparencies which could relate to the porosity of the material,

The gas permeable material may be a polymer, such as a chemically-optimised gas permeable polymer. Chemically-optimised polymers may be advantageous over corresponding unmodified polymers because they may be cheaper, more resistant to tear, hydrophobic, antistatic, more transparent, easier to fabricate with, less brittle, more elastic, more permeable to gases and selectively permeable to specific gasses, Chemical modifications on polymers may be performed in any way a skilled person will know such as by modifying the chemical composition of the monomer, the back bone chain, side chains, end groups, and/or the use of different curing agents, crosslinkers, fillers, processes of vulcanisation, manufacture, fabrication, and other methods.

The membrane layer can comprise any suitable gas permeable material including, but not limited to: silicones, polysiloxanes, polydimethylsiloxanes (PDMS), fluorosilicone, organosilicones, VMQ (Vinyl Methyl Siloxane), PVMQ (Phenyl vinyl methyl siloxane), silicon-oxide polymers, sulfonated polyetheretherketone (SPEEK), poly(ethylene oxide), poly(butylene terephthalate), or poly(ethylene oxide), poly(butylene terephthalate) block copolymers (PEO-PBT), for example 1000PEO40PBT60; cellulose (including plant cellulose and bacterial cellulose), cellulose acetate (celluloid), nitrocellulose, and cellulose esters. Porous materials, in particular nanoporous silicon, porous silicon nanostructures are also contemplated for use.

In a suitable embodiment, the membrane layer comprises polysiloxanes, optionally optimised polysiloxanes. The polysiloxanes may be chemically-modified or machine-modified, Typically, the membrane layer comprises polysiloxane elastomers. It has been found that polysiloxanes are good candidates for gas permeable membranes thanks to the Si—O bonds into the polymer structure which facilitates higher bond rotation, increasing chain mobility, and thereby increasing levels of permeability. Polysiloxane elastomers (such as silicone rubber) are also flexible, tolerant to UV radiation and resilient materials.

In an embodiment, the membrane layer comprises polydimethylsiloxanes (PDMS), suitably optimised polydimethylsiloxanes. Typically the membrane layer comprises polydimethylsiloxane (PDMS) elastomers. Polydimethylsiloxanes (PDMS) can take form of an elastomer, a resin, or a fluid. The PDMS elastomer can be formed by using a cross-linking agent, by UV curing techniques and other methods. PDMS is a typical gas permeable material because of its very high oxygen, carbon dioxide and water vapour permeability, its optical transparency and its tolerance to UV radiation. These elastomers typically do not support microbiological growth on their surface, and so avoid uncontrolled biofilm growth and/or biofouling which can reduce the efficacy of the device to generate biomass (shielding light). Optionally a biofilm growth can be facilitated by utilising biological supports and/or additional components as described below. Additionally, polydimethylsiloxanes (PDMS) elastomers are flexible and resilient materials.

The polydimethylsiloxanes (PDMS) may be chemically-modified or machine-modified to increase its gas permeability and/or to change its properties. PDMS elastomers typically have an oxygen permeability of at least 350, at least 400, at least 450, at least 550, at least 650, at least 750, suitably at least 820 Barrers. Suitably the carbon dioxide permeability of PDMS elastomer is at least 2000, at least 2500, at least 2600, at least 2700, at least 2800, at least 2900, at least 3000, at least 3100, at least 3200, at least 3300, at least 3400, at least 3500, at least 3600, at least 3700, at least 3800, suitably at least 3820 Barrers. The properties of the PDMS used in embodiments of this invention can be optimised through chemical, mechanical and process-driven interventions related to but not limited to the molar mass (Mₘ) of polymer chains, the dispersity in the polymer (dispersity is the ratio of the weight average molar mass to number average molar mass), the temperature and duration of the heat treatment during curing, the ratio of the cross-linking agent to PDMS, the cross-linking agent chemical composition, different end groups (such us methyl-, hydroxy- and vinyl-terminated PDMS) which can influence the way in which end-linked PDMS structures form during cross-linking.

Alternatively, nanocomposites could be used for making highly gas-permeable membrane materials. Nano-materials and nano-structures mixed together with a membrane material can be used to increase permeability of that membrane material. Nano-clay filled siloxanes and more specifically nano-clay filled poly (dimethylsiloxane) PDMS are examples which could be used in the present invention. It was found that nanoclay (nanoparticles of layered mineral silicates) provides substantial polymer reinforcement, though the gas permeability of the nanocomposite remains high, despite the large nanolayer aspect ratio. The random orientation of the clay nanolayers in the polymer matrix is responsible for the lack of an effective gas barrier property, thereby increasing its gas permeability properties.

In another embodiment, the membrane layer comprises bacterial cellulose. While bacterial cellulose has the same molecular formula as plant cellulose, it has significantly different macromolecular properties and characteristics. In general, bacterial cellulose is more chemically pure, containing no hemicellulose or lignin. Furthermore, bacterial cellulose can be produced on a variety of substrates and can be grown to virtually any shape, due to the high moldability during formation. Additionally, bacterial cellulose has a more crystalline structure compared to plant cellulose and forms characteristic thin ribbon-like microfibrils, which are significantly smaller than those in plant cellulose, making bacterial cellulose much more porous. The skilled person will be aware of a number of bacterial systems that are engineered to optimise cellulose production, such as the cellulose biosynthetic system of Acetobacter sp., Azotobacter sp., Rhizobium sp., Pseudomonas sp., Salmonella sp., and Alcaligenes sp., which can be expressed in E. coli, for example. Bacterial cellulose can be treated such that its surface provides a chemical interface to enable bonding with molecules.

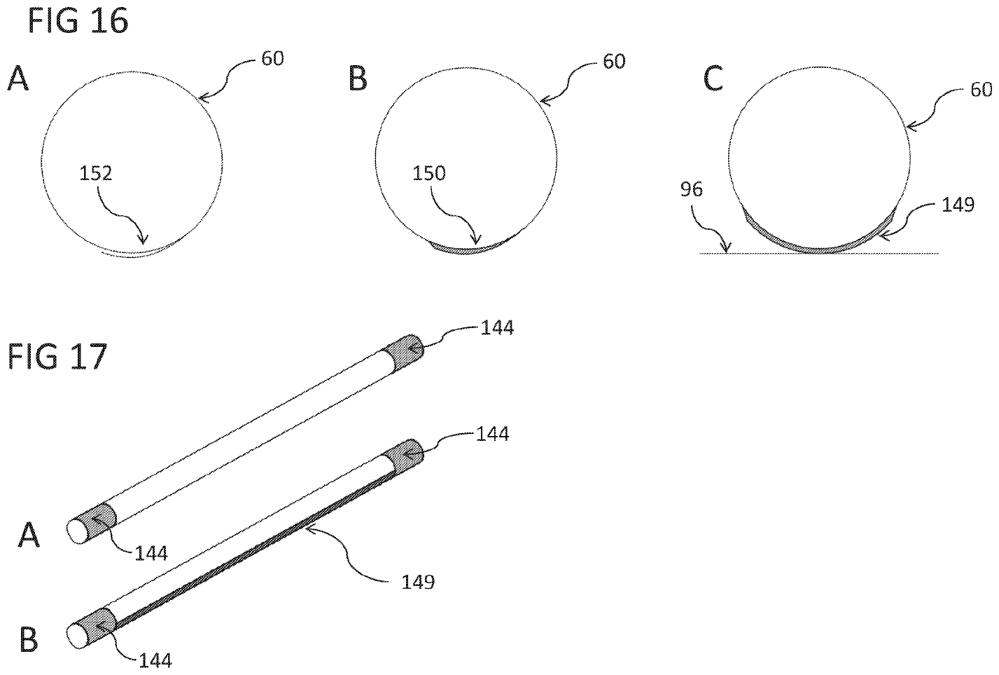

Other layers of the bioreactor may also be a membrane layer—i.e. gas permeable layer—as defined above, or they may be comprised of a non-membrane layer, comprising any suitable material, such as a natural or synthetic material. Suitably, the layers are at least translucent, and are typically transparent. The layers are suitably breathable. In a typical embodiment, all layers of the bioreactor are gas permeable membrane layers as defined herein. In other embodiments, the membrane bioreactor comprises a single layer, such as a tube or a single membrane formed of a continuous layer or a single layer folded on and sealed to itself in one or more places to create the bioreactor. For example as shown by the transverse section of FIGS. 16a and 16b, the single layer is folded on itself to form a bioreactor (60) and the area where the two edges of the same layer overlap (152) are sealed together with a glue adhesive to form a seam (150).

The membrane layers may be made substantially entirely of the gas permeable material, or may comprise additional materials. In particular, the membrane layers may have one or more integral ribs, or may comprise an internal mesh, which may be made of a support material, which is typically strong and rigid or semi-rigid, and may be flexible and/or elastic. Suitably, the support material can be flexible but not elastic, for example to allow the bioreactor to be shaped in a particular way. These structures can provide the bioreactor with improved strength and/or aid in the bioreactor holding its shape, and are arranged such that the membrane as a whole remains permeable to gases. Such internal materials may for example be the result of coextrusion of the gas permeable material and the support material.

Suitably, the bioreactor comprises a tube, pipe or hose, typically with an axial length in excess of its luminal width (i.e. diameter), comprising a single continuous membrane of gas permeable material, which may be made by extrusion, moulding, injection moulding, from a single membrane layer folded on and sealed to itself and rotational moulding or by any other appropriate process. Typically, such a tube or hose arrangement has a substantially uniform cross-section bore across at least the majority of its length, optionally for the entirety of its length. This cross-section profile may be (but does not have to be) round or circular, or may be elliptical, ovoid, or in the shape of a rounded off polygon, such as a square or rectangle. Suitably, the cross-section lacks internal blind endings, sharp corners, edges, seams and other crevices. In other words, for at least the majority of the length of the bioreactor, the interior profile of the bore of the bioreactor is substantially uniform with a smooth surface. End-reinforcements (144) can be used to reinforce the terminal portions of the membrane hose section by having a thicker wall or stronger material attached (FIGS. 17a & 17b). This is to reinforce the areas where the hose comes into contact with the connector to connect it to the adjacent hose section. Similar reinforcements can be applied along the underside of the hose section (149) (bottom-reinforcements), especially if the hose is resting on a flat or planar surface, cradle or support mesh (FIG. 17b and cut section FIG. 16b). This is to reinforce the the underside seam and avoid tears and punctures while contacting supporting surface as well as during installation. In other embodiments the reinforcement underside (149) can coincide with the seam position, where the single membrane layer is folded on and sealed to itself (152 in FIG. 16a); suitably the reinforcement underside (149) comprises a glue adhesive used to seal the single membrane layer to itself to form an elongated hose bioreactor (FIG. 16c). This reinforcement can be done in any suitable way, for example by attaching thicker layers of the same membrane material (using adhesive methods), or attaching a stronger and/or thicker material for example a flexible non-elastic polymer or a thicker mesh, or by using more layers of thermo curing silicone adhesive tapes, or by using more layers of self-curing (or UV curing) silicone glue to make a thicker layer.

In a suitable embodiment, the first and second layers, or a single layer folded on itself to form a bioreactor (suitably a hose bioreactor), are bonded by adhesion and/or heat pressing. Heat pressing utilises the application of heat and pressure for a pre-determined period of time so as to form a weld. The skilled person in the art will be familiar with suitable heat pressing techniques for this application. The precise temperature and duration required to bond portions of the first and second layer's together will depend on the specific materials comprised in the two layers. Alternatively or additionally, a glue interface can be used to bond portions of the two layers together or a single layer folded on itself; once applied on the layers or on the single layer the glue interface can be cured utilising heat pressing techniques, or can cure spontaneously at room temperature, or can cure spontaneously at specific temperatures, or can cure after being irradiated with UV light (a light comprising of ultra violet wavelengths) or other suitable light wavelengths, or can cure using heat or pressure alone. As used herein, the term “glue interface” also includes the use of non-crystallised (non-vulcanised) polymers that can bond the two layers with heat or humid pressing. As used herein, the related terms, “glue interface”, “adhesive” and “adhesive interface” are synonymous, and the three terms can be used interchangeably herein.

The glue interface thickness varies depending on its composition, material and the layer material. Suitably, the glue interface thickness is no less than: 1 μm, optionally 10 μm, suitably 20 μm, typically 50 μm. Typically, the glue interface thickness is no more than 20 mm, no more than 10 mm, no more than 5 mm, no more than 2 mm, optionally 1 mm, suitably 600 μm, typically 200 μm.

More specifically, if the first and second layers or a single layer folded on itself are comprised of polysiloxanes and/or dimethylpolysiloxanes (PDMS), the two layers can be bonded together by using silicone adhesives which can be in liquid form, viscous liquid gel form, a layer form, a layer tape form, and/or may comprise all types of silicone adhesive which can cure below or above 22° C. or can cure with pressure, or can cure after being irradiated with UV light (a light comprising of ultra violet wavelengths) or other suitable light wavelengths. After applying the silicone adhesive on both layers or a single layer folded on itself, the bonding areas are typically pressed for a determined period of time as dictated by the type of silicone adhesive and, if the type of silicon adhesive used also needs heat to cure, it is heated at a determined temperature and for a determined period of time as dictated by the type of silicone adhesive which is utilised.

Types of possible silicone adhesives include, but are not limited to, silicone glues and silicone adhesive layers such as the VVB Birzer ADT-X (which bonds with heat pressing for 30 to 60 seconds at pressures between 1 and 15 N/cm² and temperatures between 140 and 180° C.) with thicknesses between 0.20 mm and 0.60 mm, the Adhesives Research Arclad® IS-7876 silicone transfer adhesive (which is a pressure-sensitive adhesive which bonds with pressure and temperatures above ˜5° C.) with thicknesses between 25 and 100 μm, the Techsil® RTV10533 one-component silicone adhesive that cures when exposed to atmospheric moisture at room temperature.

Alternatively the silicon adhesive interface can be composed of a thin layer of un-cured polysiloxane and/or dimethylpolysiloxane (PDMS), which can be mixed with its cross-linking agent, and quickly applied on the intended bonding regions on the layers, then pressed and heated to cure, bonding the two layers together.

In some embodiments, the “glue interface” and/or silicone adhesive can be used to bond the two layers together or a single layer folded on itself in the region where the fluid conduit is typically located. This bonding will create a control structure to control the flow of the liquid media, dividing or diverting the fluid conduits in multiple conduits.

Advantages of embodiments with one or more bioreactors which are in the shape of a tube or hose include the reduction of sites within the bioreactor where liquid media, cells and/or contaminants can accumulate, due to the substantially uniform cross-section and lack of internal edges, seams, crevices and suchlike. In narrow, restricted internal places such as internal seams, flow rate could be reduced, and solid objects such as cells or contaminants could be trapped or otherwise accumulate. Such restricted places are also difficult to clean effectively, as cells, debris and contaminants can become stuck. This could lead to cell breakdown and further contamination of the bioreactor contents.

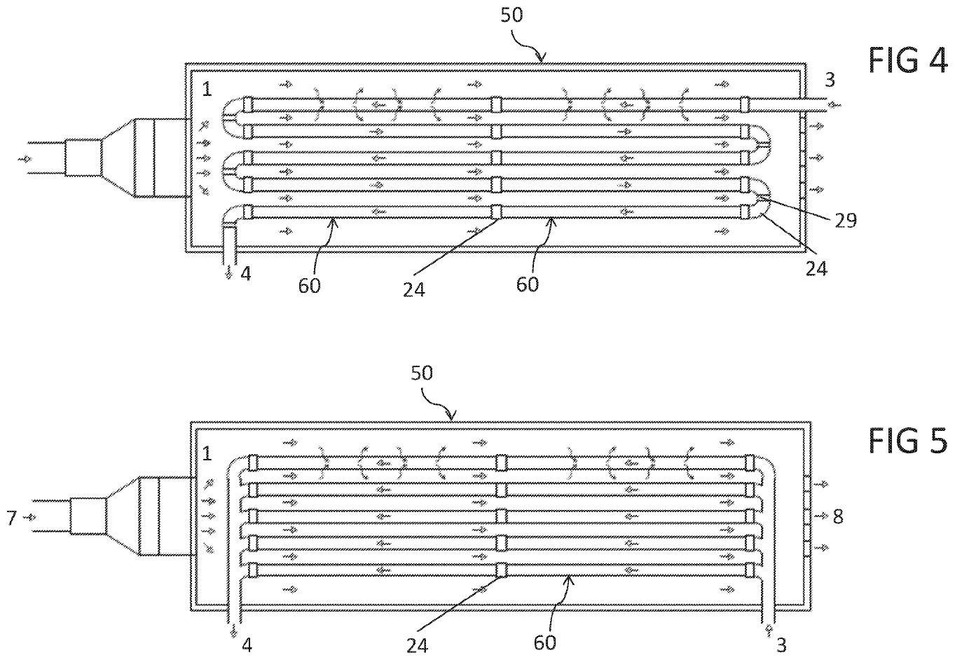

Tube or hose arrangements are also space-efficient, and multiple tube bioreactors can be arranged within a single chamber, in series, where the outlet of one bioreactor flows into another bioreactor to which it is connected (see for example FIG. 4), in parallel (see for example FIG. 5), or in a combination of these approaches. For example, multiple tube bioreactors may be arranged in series such that the flow within each bioreactor runs in an antiparallel direction to the preceding one, such that the liquid media takes a sinuous path through several bioreactors. Where two or more bioreactors are connected so as to be in fluid communication with each other, the connector or conduit which joins them can be a separate component, which does not have to comprise any gas permeable materials. Connectors may also be used to connect bioreactors to the auxiliary system or to an outlet or inlet. The connector may comprise a valve, typically a solenoid valve or diaphragm valve, which acts to prevent or allow fluid passing through the connector, for example between one bioreactor and the next. Advantageously, this can allow for several ‘blocking points’ within a system comprising multiple bioreactors arranged in series. This enables any hydrostatic pressure stress from abruptly halting flow within the system to be shared between adjacent bioreactors, and to prevent pressure waves from propagating throughout the whole of the connected bioreactors. Otherwise, if the flow is stopped suddenly, such as due to a pump failure, with all bioreactors remaining fluidly connected, a ‘water hammer’ effect may put excessive stress on particular components within the system. Any measures to mitigate such effects may be used in systems according to the invention, as appropriate, such as pressure regulators, slow-closing valves, flow diverters, shock absorbers, dampeners, and so on.

It is contemplated that features may be introduced that allow for improved mixing of the liquid media as it flows through the bioreactor or bioreactor array. In this regard, static mixers can be installed in the bioreactor (either inside the membrane bioreactor itself, or inside one or more connectors between membrane bioreactors) to increase turbulence in the bioreactor and facilitate mixing of liquid culture. These mixers are static and designed to mix a fluid in motion that passes through them. For instance, a static mixer can comprise a helicoidal structure which disrupts the flow of liquid media.

The gas permeable membranes may be no more than about 2000 μm in thickness, no more than about 1000 μm in thickness, suitably no more than about 800 μm, about 600 μm, about 500 μm, about 400 μm, about 200 μm and typically no more than about 100 μm, optionally no more than about 50 μm, suitably no more than 20 μm, suitably no more than 10 μm or less. The gas permeable membranes may be at least 10 μm in thickness, at least 20 μm in thickness, suitably at least 50 μm, at least 100 μm, at least 200 μm and optionally at least 500 μm in thickness. The thickness of the bioreactor membrane may vary across its length, for example where a bioreactor is connected to another bioreactor or another object by a connector, the thickness may be increased in a portion of the membrane proximate to the connector compared to the membrane distant to the connector. Membrane thickness can also change depending on the position of the bioreactor in the array, for example bioreactors in a lower vertical position may be thicker, to provide more protection against swelling under pressure.

The diameter of the bioreactors of the invention (that is, the largest diameter of the cross section of the bioreactor perpendicular to the direction of liquid media flow), may be no more than about 20 cm, no more than 15 cm, 10 cm, 9 cm, 8 cm, 7 cm, 6 cm, 5 cm, 4 cm, 3 cm, 2 cm, or no more than about 1 cm. The diameter may be no less than about 0.5 cm, no less than about 1 cm, 2 cm, 3 cm, 4 cm, 5 cm, 8 cm, or no less than about 10 cm. Typically the diameter is between 8 cm and 2 cm, typically between 7 and 2 cm, suitably between 5 and 3 cm. The diameter may be typically below 5 cm for chemoheterotrophs and below 10 cm for photoautotrophs.

The length of the bioreactor, being the distance between the inlet and the outlet of a single bioreactor, may be no more than about 100 m, optionally no more than about 75 m, about 50 m, about 25 m, about 10 m, about 9 m, about 8 m, about 7 m, about 6 m, about 5 m, about 4 m, about 3 m, about 2 m, about 1 m, about 0.5 m, typically no more than about 0.1 m. Typically the length of a single bioreactor is between about 10 m and about 1 m, suitably between 5 m and 1 m, and in an embodiment between 3 and 1 m.

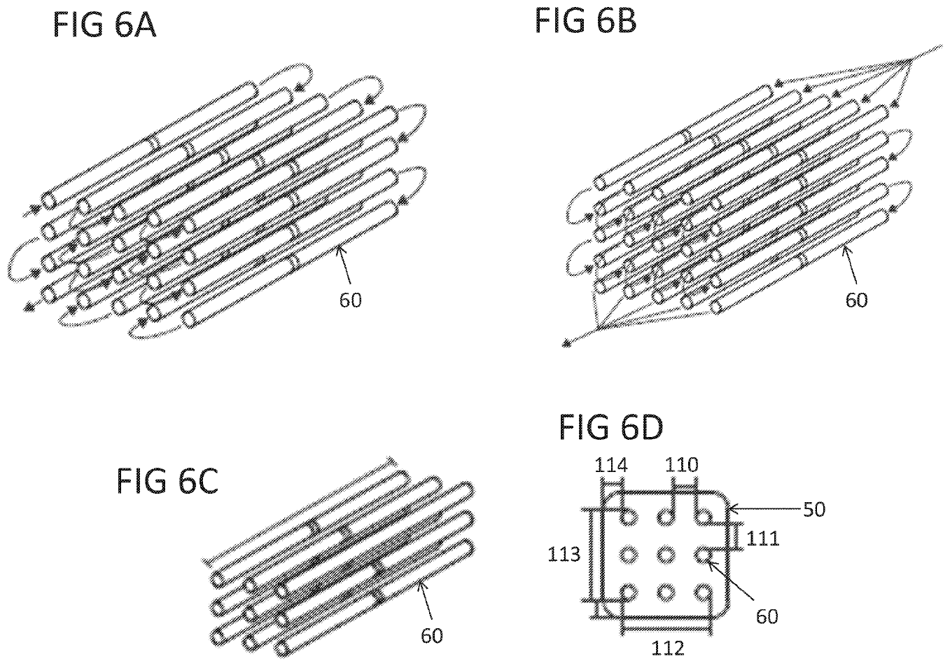

As discussed, multiple bioreactors can be connected in series, and can be arranged such that the flow direction of one bioreactor is opposite to the flow direction of the preceding bioreactor. The length for which consecutive bioreactors can be arranged to run before such a change of direction occurs can be no more than about 2000 m, 1500 m, 1000 m, 750 m, 500 m, 400 m, 300 m, 250 m, 200 m, 100 m, 80 m, 60 m, 40 m, 20 m, 10 m, 5 m, 1 m or less. Suitably this length is between about 1000 m and about 50 m, typically between about 800 m and about 150 m, suitably between about 400 m and about 200 m, optionally between about 300 m and about 100 m. Generally, this length is selected to be as long as possible before a change in direction occurs (as this causes pressure increases) but without causing undue difficulties in maintenance.

Where multiple bioreactors are arranged horizontally, due to bioreactors connected in series changing in direction, multiple bioreactors being arranged in parallel, or otherwise, the horizontal (width) dimensions of the array of bioreactors (see FIG. 6D) may be no more than about 200 m, 150 m, 100 m, 75 m, 50 m, 40 m, 30 m, 25 m, 20 m, 15 m, 10 m, 9 m, 8 m, 7 m, 5 m, 4 m, 3 m, 2 m, suitably no more than about 1 m or less. Suitably this dimension is between about 75 m and about 1 m, typically between about 40 m and about 5 m, optionally between about 30 m and about 5 m, and suitably between about 20 m and about 8 m. The minimum horizontal dimension can evidently be no less than the horizontal diameter of a single bioreactor. This width dimension should be chosen to allow sufficient volume of liquid media to be contained, but not to be so wide that excessive pressure is created through the need for multiple changes of flow direction.

Similarly, multiple bioreactors can be arranged or ‘stacked’ vertically. The minimum height of an array of bioreactors can evidently be no less than the height of a single bioreactor. The total height of an array (see FIG. 6D) may be no more than about 100 m, 50 m, 25 m, 20 m, 10 m, 9 m, 8 m, 7 m, 6 m, 5 m, 4 m, 3 m, 2 m, 1 m, 0.5 m, 0.4 m, 0.3 m, 0.2 m, typically no more than about 0.15 m. Typically, this dimension is between about 10 m and about 0.15 m, suitably between about 5 m and about 0.5 m, optionally between about 3 m and about 0.5 m, alternatively between about 2 m and about 1 m. Height should be chosen to allow sufficient volume of liquid media to be contained, but not to be so high that excessive pressure is created, and/or to cause difficulties in maintenance.

Where multiple bioreactors are arranged side-by-side or vertically, the gaps left between them, vertically or horizontally (see FIG. 6D), may be at least about 1 mm, about 5 mm, about 10 mm, about 50 mm, or at least about 100 mm. Typically, the gap is about 10 mm horizontally, and suitably about 50 mm vertically. In some situations, no gap may be left (that is, neighbouring bioreactors may touch). In general gap size is chosen to allow gas to circulate effectively between bioreactors.

The volume comprised within the bioreactors or arrays is not intended to be particularly limited except by the capacity of the bioreactors and other parts of the system.

The Chamber

The chamber is typically defined by one or more exterior walls, and comprises a gas mixture that may include O₂, such as, for example, atmospheric air. The concentration of O₂ in the gas mixture may be higher than that comprised within the liquid media within the bioreactor, thereby increasing the concentration differential between the liquid media and the surrounding atmosphere within the chamber. In this way the gas-transfer rate of O₂ through the membrane into the liquid media is increased.

As the O₂ in the liquid media is consumed by the cells comprised within, and more O₂ passes across the membrane of the bioreactor from the atmosphere within the chamber to the liquid media, the O₂ gas transfer rate will decrease over time as the concentration differential stabilises to an equilibrium state. To overcome the tendency toward equilibrium, the gas mixture comprising O₂ can be continuously or intermittently delivered through a gas chamber inlet, and a similar volume of gas can be removed through an outlet, typically using a controlled valve such as a solenoid valve and/or a pressure sensitive valve. Optionally the valve can be closed and/or restricted when the gas mixture is delivered, to pressurise the gas chamber above ambient standard atmospheric pressure and so further increase gas transfer rate across the gas-permeable membrane of the bioreactor.

The gas mixture introduced into the gas chamber may also comprise a lower concentration of CO₂ than that found in the liquid media of the bioreactor and/or than atmospheric CO₂ levels, in order to increase the CO₂ depletion rate from the liquid media. Alternatively, CO₂ can be removed from the liquid media by the introduction into the gas chamber of inert gases such as nitrogen, helium, argon or methane and/or O₂ in order to increase the CO₂ concentration differential between the atmosphere and the liquid media. It may also be desired to increase the concentration of CO₂ in the gas mixture. For example, CO₂ or other gases may be used to change the pH level of the liquid media. This can be beneficial to encourage the growth of organisms which prefer low pH, such as so-called extremophiles, some of which can grow in environments with a pH of between 2 and 4. Additionally, certain organisms react to the stress of a low pH environment by changing their behaviour and/or biomass production, and it may be desired to stimulate production of a particular stress-induced product.

Other organisms may require the supply of different gas, and the chamber atmosphere can be controlled accordingly, for example CO₂ can be supplied where the organisms are autotrophic, methane can be supplied where the organisms are methanotrophic, or hydrogen where the organisms are hydrogen oxidising organisms or hydrogenotrophic organisms. Certain hydrogen oxidising organisms are defined by the ability to use gaseous hydrogen as an electron donor with oxygen as electron acceptor and to fix carbon dioxide. As a result a chamber atmosphere comprising a mix of hydrogen, carbon dioxide, and O₂ could be used in the chamber. These “CO₂ dependent” hydrogen-oxidising organisms contrast with those (such as Acetobacter, Azotobacter, Enterobacteriaceae, and others) that also oxidise hydrogen under aerobic conditions, but cannot carry out autotrophic carbon dioxide fixation. Where hydrogen is supplied, it is contemplated that electrolysis to produce hydrogen from water can be carried out in the auxiliary system, for example directly inside the liquid media or in a water tank in or next to the chamber, which would, avoid pumping hydrogen into the gas chamber, which may have safety implications,

Equally, anaerobic conditions may be preferred by certain organisms, such as certain hydrogen oxidising organisms and methanogens. In this case, the chamber atmosphere can be controlled to lack oxygen, or any gas which could be detrimental to growth and/or survival.

In some embodiments, the gas chamber may be separated into two or more sections, referred to herein as first and second chambers etc., into which different gases or gas mixtures can be introduced. For example, the first chamber can contain an O₂-enriched gas mixture, while the second may contain a CO₂-depleted gas mixture such as N₂-rich gas for the effective removal of CO₂. In certain embodiments of the invention the bioreactor provides an intervening barrier between the first and second chambers (and further chambers if required). Hence, in this embodiment of the invention the first and second chambers are defined by exterior walls of the chamber in combination with the membrane wall of the intervening bioreactor.

The gas can be moved inside the chamber passively by gas expansion, or by using a low energy method which reduces O₂ (or any other suitable gas) feed delivery costs such as a fan, turbine or other impeller. Alternatively, the gas can be compressed prior to introduction into the gas chamber. It is contemplated that the pressure inside the chamber can be controlled by the introduction or removal of gas. For example, the pressure inside the chamber can be higher than atmospheric pressure outside the chamber, or else pressure inside the chamber can be reduced compared to the atmospheric pressure outside the chamber.

The internal environment of the chamber can be controlled internally or by controlling the gas supply and/or the gas discharge. For example, the humidity of the atmosphere within the chamber can be controlled by introducing a gas mixture with reduced or increased humidity compared to the chamber atmosphere, or by the presence of a desiccating or humidifying agent installed in the gas inlet, or by a desiccating or humidifying agent or material or coating placed inside the chamber itself or within an attached auxiliary system. Most commonly, the chamber atmosphere requires desiccation, due to water vapour passing from the liquid media through the bioreactor membrane into the chamber atmosphere. For example the chamber atmosphere can be circulated to a dessicant for drying, before being returned to the chamber; typically the desiccant can be in the form of a honeycomb wheel. For example the temperature of the chamber atmosphere can be controlled by introducing a gas mixture with reduced or increased temperature compared to the ambient chamber atmosphere, or by the presence of a cooling or heating component installed in the gas inlet and/or before the gas inlet. For example the chamber atmosphere can be circulated to an air conditioning unit and/or an air heating unit, before being returned to the chamber. In some cases, the gas mixture in the chamber can be recirculated in the same chamber, or passed to the next chamber in cases where multiple chambers are arranged in series. Before returning a gas mixture to a chamber, the gas can be desiccated, cooled, heated, filtered, cleaned and/or replenished with a suitable amount of desired gas to adjust its composition and/or be cooled, heated, and/or desiccated further.

The internal chamber temperature can also be controlled or influenced by controlling the temperature of the gas introduced into the chamber. For example, heated or cooled gas can be introduced which can control the temperature of the chamber atmosphere and even the liquid media of the bioreactors. Heating and/or cooling units can be comprised by or contained within the chamber itself, which can control the temperature of the atmosphere already within the chamber more directly.

At least a portion of the walls that define the chamber material may be transparent or translucent, to allow the effective transmission of light such that when the cells comprised within the bioreactor are phototrophic or mixotrophic, they can use the light for the production of energy or the fixation of inorganic carbon. Such transparency may also be useful even where the cells do not require light, for example to enable straightforward inspection of the chamber interior by an operator. In some embodiments, at least a portion of one or more of the walls, for example the wall located furthest from a light source, is reflective, in order to increase the passage of light through the bioreactor. In some embodiments, at least about 10%, at least about 20%, at least about 30%, at least about 40%, at least about 50%, at least about 60%, at least about 70%, at least about 80%, at least about 90% or at least about 100% of the area of the walls may be permeable to light.

‘Switchable glass’, ‘Smart glass’ or similar materials may be used in the invention. These are materials (which can be but are not limited to being rigid like glass, flexible like a polymer film or a coating) whose light transmission properties are altered when voltage, light or heat is applied. These may be of particular use in areas with high light exposure, for example to reduce damage to the materials or the microorganisms as a result of especially high light. Typically, the material changes from substantially translucent, and/or with a reflective optical property (similar to a mirror finish) to substantially transparent, changing from blocking some (or all) wavelengths of light to letting light pass through. Examples of technologies that may be used in pursuit of the above include but are not limited to electrochromic, photochromic, thermochromic, suspended particle, micro-blind and polymer dispersed liquid crystal devices.

Suitably, the walls of the chamber are substantially gas-impermeable and the chamber as a whole is substantially air-tight, to prevent loss or contamination of the controlled atmosphere comprised within. It is not necessary for the chamber to be entirely air-tight, as long as it fulfils the purpose of allowing the atmosphere within to be controlled to some extent either in terms of gas composition, temperature, humidity, pressure or otherwise.

The walls of the chamber can be composed or defined by the structures or body assemblies of vehicles, industrial machines, ships, spaceships or spacecraft, submersible vehicles, wall cavities, containers, greenhouses, underground chambers, architectural structures, building rooms and/or switch houses.

In these and/or other cases, the chamber walls could comprise materials which are not transparent/translucent. In such cases auxiliary light sources inside the chamber may be used. These auxiliary light sources could be LEDs/OLEDs or fluorescent tubes, or could be natural light channelled by fibre optics and/or optic assemblies. Similarly in cases where the chamber walls are translucent/transparent but the device is located inside or is otherwise remote from natural light, such auxiliary light sources may be used. In some cases, at least part of the interior chamber walls may be, or may comprise, reflective material. In cases where interior light sources are used, this may increase the efficiency of light supply to the cells. In some cases, a mixture of translucent/transparent and reflective material may be used, for example where an external light source is used. In some such instances, part or all of the interior wall or walls furthest from the light source may be reflective, to increase the efficiency of use of the supplied light. In embodiments where mixotrophic organisms are cultured, the light sources may supply the light necessary for their growth. The light sources may be configured to provide sporadic and/or intermittent illumination, depending on the requirements of the embodiment of the invention and/or the organisms used.

Any translucent/transparent portion which permits transmission of light into the chamber can be composed of any suitable translucent/transparent material. The chambers can be comprised entirely of the translucent/transparent material, or can be supported on a support structure such as a scaffold or frame, as discussed below.

Suitably the chamber is comprised of substantially gas-impermeable material that is strong, light, and that may possess good thermal insulation properties. Optionally the material is provided in sheets and/or films. In some embodiments the material is non-flexible, non-elastic, transparent and strong, for example comprising glass, high performance glass, low iron glass with very high solar energy transmittance (Pilkington Sunplus™), glass composites, reinforced glass composites with increased strength, impact proof glass composites, low reflectance glass, high light transmittance glass, double glazing style glass and/or triple glazing with or without vacuum/argon/air in between, or glass composites made of several layers of different materials to increase strength and/or light transmittance, or electrically switchable smart glass. Alternatively, the chamber may be comprised of a metal or metal alloy, such as aluminium or steel, or of a composite material such as carbon fibre composite, fibre-glass, or wood fibre materials (e.g. MDF), concrete, stones, clay, ceramic tiles, tiles, plaster, plastic polymers,

In other embodiments the chamber wall material is flexible and elastic, for example comprising ethylene tetrafluoroethylene (ETFE), acrylic/PMMA, polycarbonate and/or other plastics and plastic composites. Suitably, the chamber wall material comprises polyvinyl chloride (PVC), polyurethane, vulcanised rubber, silicones, a polyvinyl, and/or nylon, textile-reinforced urethane plastic, woven fabrics coated with polymers such as PVC, Nylon, PC, silicone, rubber.

The suitable properties of ETFE include its translucency and/or transparency, very high light transmittance, and ultraviolet resistance. ETFE is also advantageously recyclable, easily cleanable (due to its non-adhesive surface), elastic, strong and light, with good thermal insulation, high corrosion resistance and strength over a wide temperature range. Employing heat welding, tears can be repaired with a patch or multiple sheets assembled into larger panels.

Acrylic is suitable as chamber wall material due to its strength, high transparency, and resistance to weathering and ultraviolet radiation.

In specific embodiments of the invention use of flexible and/or elastic material allows for the chamber to be inflated by supplying an atmosphere within the chamber that has a relative positive pressure compared to the surrounding atmosphere outside of the device. Alternatively, gas expansion within the chamber due to an increase in temperature may also cause a corresponding increase in relative positive pressure. In some embodiments the pressure in the chamber can even be negative compared to the surrounding atmosphere outside of the device, for example by the action of fans or blowers removing gas out of the chamber. The chamber can be entirely inflated from a collapsed (uninflated) state, and/or can be built around or otherwise supported by a rigid or semi-rigid scaffold, which may be internal or external to the chamber itself, and may be integral to the chamber, or separable from it. The chamber wall material can be reinforced by the inclusion of an integral skeleton of members of a rigid or semi-rigid scaffold, and/or by the use of reinforcing seams made from the same or similar material to the chamber walls. These reinforcements can also be used to control the shape and structure of the chamber when constructed and inflated. Such arrangements allow for systems according to some embodiments of the invention to be easily and rapidly constructed, taken down, and/or transported in their collapsed (uninflated) forms. Weight can also be reduced by use of such embodiments, increasing suitability for transportation, and for temporary and/or remote usage, such as in space, polar research stations or other inaccessible locations. Such portable structures can also be put up inside warehouses or any kind of structure or chamber, such as underground chambers or tunnels, in order to create multiple independent chamber modules inside a structure which offers protection from the environment. These inflated chambers can be easily changed, disassembled or moved to update the array of the bioreactors without compromising the structure of the building.

In specific embodiments of the invention the use of flexible and/or elastic materials will allow to create a convex, domed, cambered, or otherwise protuberant shape to the upper wall of the chamber (relative to a position outside the chamber) either as a result of positive pressure inside the chamber relative to the surrounding atmosphere (that is, inflation of the chamber by the gas supplied) or by using auxiliary structures attached to the walls of the chamber, to create the convex shape. This can be helpful to avoid the formation of “puddles” of rain, snow, leaves, powder, sand or other detritus if the apparatus is deployed in the field. Moreover the convex shape will facilitate the self-cleaning of the material when raining and/or facilitate manual/automatic cleaning performed by the plant operators or automatic cleaning system. For similar reasons, in other embodiments of the invention any upper surfaces of the chamber may be tilted slightly relative to the horizontal, for example by having side walls of the chamber of different heights.

Another advantage of such an arrangement is to enable a measure of control over internal chamber humidity—moisture in the chamber atmosphere may condense on the inside of chamber walls, especially if the inside of the chamber is warmer than the outside atmosphere. With convex or tilted upper walls any condensation can be encouraged to run away from the upper walls of the chamber, reducing the interference on light transmission that might occur.

Graphene coatings may be used to reinforce the material, to provide antimicrobial growth coatings, to provide electrical conductance that can then help detect breakages (e.g. tearing) of the material. Coatings, treatments, paints or films to reduce mould, bacteria and fungi growth can also be applied to the inside surface of the chamber. Specific materials intended to prevent mould or any microbial growth can be used as components of the chamber. The material can also comprise graphene, carbon nanotubes and/or graphite for reinforcement, or to enable a thinner and lighter wall material to be used.

It is envisaged that the inside of the chamber may be easily accessed for maintenance purposes by full or partial removal of one or more of the walls that comprise the chamber.

The minimum dimensions of the chamber are largely dictated by the size of the bioreactor or bioreactor array contained. In some embodiments, sufficient additional space may be left between the outermost edges of the bioreactor or bioreactor array and the chamber walls to allow for the access of maintenance personnel or equipment (see FIG. 6D).

The Organisms

The devices and methods of the inventions may be used to culture any microorganism, cell or small organism taken from Bacteria, Archaea or Eukaryota taxonomy domains, as long as it can be supported in a suitable liquid medium. Such cells and organisms can be heterotrophic or mixotrophic. Additionally, the devices and methods of the inventions are suitable for culturing phototrophic organisms, including photoautotrophic organisms.

More specifically, the cells and/or organisms can be part of the taxonomic groups and other defined groups including the following: Cyanobacteria, Protobacteria, Spirochaetes, Gram Positive bacteria, green filamentous bacteria such as Chloroflexia, Planctomycetes, Bacteroides cytophaga, Thermotoga, Aquifex, halophiles, Methanosarcina, Methanobacterium, Methanococcus, Thermococcus celer, Thermoproteus, Pyrodictium, Entamoebae, slime moulds such as Mycetozoa, Ciliates, Dinoflagellates, Dinophyceae, Trichomonads, Microsporidia, Diplomonads, Excavata, Amoebozoa, Choanoflagellates, Rhizaria, Foraminifera, Radiolaria, Diatoms, Stramenopiles, brown algae, red algae, green algae, snow algae, Haptophyta, Cryptophyta, Alveolata, Glaucophytes, phytoplankton, plankton, Percolozoa, Rotifera, and cells or whole organisms from animals, fungi or plants.

Suitable Bacteria can include Escherichia coli, Escherichia coli BL21(DE3), Escherichia sp., Acetobacter sp., Acetobacter xylinum, Arcina ventriculi, Zymomonas mobilis, Gluconobacter xylinus, Pseudomonas sp. #142, Microbacterium laevaniformans, Paenibacillus polymyxa, Bacillus licheniformis, Bacillus subtilis, Bacillus macerans, Streptococcus salivarius, Leuconostoc mesenteroides, Aerobacter levanicum, Gammaproteobacteria and Alphaproteobacteria, Vibrio sp., Vibrio natriegens, Pseudomonas fluorescens, Caulobacter crescentus, Agrobacterium tumefaciens, and Brevundimonas diminuta. Other suitable bacteria can include Deinococcus sp., Deinococcus radioduran, Deinococcus geothermalis, D. cellulolysiticus, D. radiodurans, D. proteolyticus, D. radiopugnans, D. radio philus, D. grandis, D. indicus, D. frigens, D. saxicola, D. maricopensis, D. marmoris, D. deserti, D. murrayi, D. aerius, D. aerolatus, D. aerophilus, D. aetherius, D. alpini tundrae, D. altitudinis, D. apachensis, D. aquaticus, D. aquatilis, D. aquiradiocola, D. aquivivus, D. caeni, D. claudionis, D. ficus, D. gobiensis, D. hohokamensis, D. hopiensis, D. misasensis, D, navajonensis, D. papagomensis, D, peraridilitoris, D. pimensis, D. piscis, D. radiomollis, D. roseus, D. sonorensis, D, wulumudiensis, D. xibeiensis, D. xinjiangensis, D. yavapaiensis or D. yunweiensis bacterium. In particular, contemplated species include Escherichia coli, Escherichia sp, Acetobacter sp., Zymomonas mobilis, Gluconobacter xylinus, Pseudomonas sp., Microbacterium laevaniformans, Paenibacillus polymyxa, Bacillus licheniformis, Streptococcus salivarius, Leuconostoc mesenteroides, Aerobacter levanicum, Gammaproteobacteria and alphaproteobacteria, Vibrio sp., Pseudomonas fluorescens, Caulobacter crescentus, Agrobacterium tumefaciens, Brevundimonas diminuta. Deinococcus sp., Meiothermus ruber, and Oceanithermus profundus.

Pathogenic organisms can also be cultured in devices according to the invention, for example for use in vaccine production. Further bacteria which may be relevant include Bacillus subtilis, Corynebacterium glutamicum, Saccharomyces cerevisiae, Zymomonas mobilis, Agrobacterium tumefaciens, Sinorhizobium meliloti, Rhodobacter sphaeroides, Paracoccus versutus, Pseudomonas fluorescens, Pseudomonas putida, Salmonella enterica, Escherichia fergusonii, Yersinia pestis, Yersinia pseudotuberculosis, Yersinia enterocolitica, Shigella flexneri, Shigella sonnei, Shigella boydii, Shigella dysenteriae, Pectobacterium atrosepticum, Pectobacterium wasabiae, Erwinia tasmaniensis, Erwinia pyrifoliae, Erwinia amylovora, Erwinia billingiae, Buchnera aphidicola, Enterobacter sp. 638, Enterobacter cloacae, Enterobacter asburiae, Enterobacter aerogenes, Cronobacter sakazakii, Cronobacter turicensis, Klebsiella pneumoniae, Klebsiella variicola, Klebsiella oxytoca, Citrobacter koseri, Citrobacter rodentium, Serratia proteamaculans, Serratia sp. AS12, Proteus mirabilis, Edwardsiella ictaluri, Edwardsiella tarda, Candidatus Hamiltonella defense, Dickeya dadantii, Dickeya zeae, Pantoea anantis, Pantoea sp. At-9b, Pantoeo vagans, Rahnella sp. Y9602, Haemophilus parasuis, Haemophilus parainfluenzae, Pasteurella multocida, Aggregatibacter aphrophlus, Aggregatibacter actinomycetemcomitans, Vibrio cholera, Vibrio vulnificus, Vibrio parahaemolyticus, Vibrio harveyi, Vibrio splendidus, Photobacterium profundum, Vibrio anguillarum, Shewanella oneidensis, Shewanella denitrificans, Shewanella frigidimarina, Shewanella amazonensis, Shewanella baltica, Shewanella loihica, Shewanella sp. ANA-3, Shewanella sp. MR-7, Shewanella putrefaciens, Shewanella sediminis, Shewanella sp. MR-4, Shewanella sp. W3-18-1, Shewanella woodyi, Psychromonas ingraharnii, Ferrimonas balearica, Aeromonas hydrophila, Aeromonas salmonicida, Aeromonas veronii, Tolumonas auensis, Chromobacterium Violaceum, Burkholderia sp. CCGE1002, Azospirillum sp. B510, Bacillus anthracis, Bacillus cereus, Bacillus cytotoxicus, Bacillus thuringiensis, Bacillus weihenstephanensis, Bacillus pseudofirmus, Bacillus megaterium, Staphylococcus aureus, Exiguobacterium sibiricum, Exiguobacterium sp. ATIb, Macrococcus caseolyticus, Paenibacillus polymyxa, Streptococcus pyogenes, Streptococcus pneumoniae, Streptococcus agalactiae, Streptococcus mutans, Streptococcus thermophilus, Streptococcus songuinis, Streptococcus suis, Streptococcus gordonii, Streptococcus equi, Streptococcus uberis, Streptococcus dysgalactiae, Streptococcus gallolyticus, Streptococcus mitis, Streptococcus pseudopneumoniae, Lactobacillus johnsonii, Lactobacillus gasseri, Enterococcus faecalis, Aerococcus urinae, Carnobacterium sp. 17-4, Clostridium acetobutylicum, Clostridium perfringens, Clostridium tetani, Clostridium novyi, Clostridium botulinum, Desulfotomaculum reducens, Clostridium lientocellum, Erysipelothrix rhusiopathiae, Mycoplasma genitalium, Mycoplasma pneumoniae, Mycoplasma pulmonis, Mycoplasma penetrans, Mycoplasma gallisepticum, Mycoplasma mycoides, Mycoplasma synoviae, Mycoplasma capricolum, Mycoplasma crocodyli, Mycoplasma leachii, Mesoplasma florum, Propionibacterium acnes, Nakamurella multipartita, Borrelia burgdorferi, Borrelia garinii, Borrelia afzelii, Prochlorococcus marinus, Lysinibacillus sphaericus, Rhodopirellula baltica, or combinations thereof. In particular, Lactobacillus johnsonii, and Clostridium acetobutylicum are contemplated.

Methanotrophic organisms can metabolise methane as a source of carbon and energy. Use of such organisms can be useful in treatment of gas containing methane in devices according to the present invention, and can therefore have applicability against global warming, as methane is a powerful greenhouse gas. It is noted that the growth of some methanotrophic organisms may also require the provision of of carbon dioxide in the liquid media, in order to favour specific metabolic pathways and therefore growth. In this case the atmosphere maintained within the chamber can be adapted to meet the needs of the cultured organism, for example by providing carbon dioxide above normal atmospheric levels. Suitable methanotrophic bacteria or archaea can include Methylomonas 16a ATCC PTA 2402, Methylobacterium sp., Methylobacterium extorquens, Methylobacterium radiotolerans, Methylobacterium populi, Methylobacterium chloromethanicum, or Methylobacterium nodulans, Methylosinus sp., Methylosinus trichosporium OB3b (NRRL B-11,196), Methylosinus sporium (NRRL B-11,197), Methylocystisparvus sp., Methylocystisparvus (NRRL B-11,198), Methylomonas sp., Methylomonas methanica (NRRL B-11,199), Methylomonas albus (NRRL B-11,200), Methylococcus sp., Methylococcus capsulatus, Methylobacter sp., Methylobacter capsulatus Y (NRRL B-11,201), Methylococcus capsulatus (NCIMB 11132), Methylobacterium organophilum, Methylobacterium organophilum (ATCC 27,886), Methylomonas sp. AJ-3670 (FERM P-2400), Methylomicrobium sp., Methylomicrobium alcaliphilum, Methylocella sp., Methylocella silvestris, Methylacidiphilum sp., Methylacidiphilum infernorum, Methylibium sp., or Methylibium petroleiphilum. In particular, Methylococcus sp., Methylobacterium sp., Methylomonas sp., Methylococcus capsulatus and Methylibium petroleiphilum are contemplated.

So-called probiotic bacteria, archaea and fungi, which are organisms intended to be consumed live to provide health effects, include especially Lactobacillus, Bifidobacterium, Saccharomyces, Enterococcus, Streptococcus, Pediococcus, Leuconostoc, Bacillus, and can include Escherichia coli, Lactococcus, Enterococcus, Oenococcus, Pediococcus, Streptococcus and Leuconostoc species, Lactobacillus species may include Lactobacillus plantarum, L. johnsonii, L. acidophilus, L. sakei, L. bulgaricus, L. salivarius, L. acidophilus, L. casei, L. paracasei, L. rhamnosus, L. delbrueckii subsp. bulgaricus, L. brevis, L. johnsonii, L. plantarum and L. fermentum. Other intended species include Saccharomyces boulardii, Bifidobacterium bifidum, Bacillus coagulans, Bifidobacterium infantis, B. adolescentis, B. animalis subsp animalis, B. animalis subsp lactis, B. bifidum, B. longum, B. breve, Lactococcus lactis, Enterococcus faecium, Enterococcus durans and Streptococcus thermophilus, B. subtilis, and B. cereus. In particular, the Lactobacillus species, Bifidobacterium bifidum, Bacillus coagulans, Bifidobacterium infantis, B. adolescentis, Bifidobacterium bifidum and Bacillus coagulans, Bifidobacterium infantis, Enterococcus faecium, and Streptococcus thermophiles are contemplated.

Archaea taxonomy groups and species that can be used in the invention include in particular Crenarchaeota, Euryarchaeota, Desulfurococcales, Sulfolobales, Archaeoglobales, Halobacteriales, Methanobacteriales, Methanococcales, Methanopyrales, Thermococcales, Thermoplasmales, Aeropyrum pernix, Sulfolobus solfataricus, Sulfolobus tokodaii, Sulfolobus shibatae, Archaeoglobus fulgidus, Halobacterium sp., Metallosphera sedula, Methanobacterium thermoautotrophicum, Methanococcus jannaschii, Methanosarcina acetivorans, Methanopyrus kandleri, Pyrococcus horikoshii (shinkaj), Pyrococcus abyssi, Pyrococcus furiosus, Thermococcus litoralis, Thermococcus barosii, Thermoplasma acidophilum, Thermoplasma volcanium, Halobacterium sp. NRC-1, Methanococcus jannaschii DSM 2661, Pyrococcus abyssi GE5, Thermoplasma acidophilum DSM 1728, and Thermoplasma volcanium GSS 1.

Devices according to the invention can also be used to culture hydrogen oxidizing organisms that oxidize hydrogen as a source of energy with oxygen used as a final electron acceptor. Some of these organisms are preferably grown under microaerophilic conditions, that is, in environments containing lower levels of oxygen than present in normal atmosphere. As a result, a chamber oxygen concentration of lower than 21% O₂, typically around 2 to 10% O₂, can be maintained. For example, a mixture of hydrogen, carbon dioxide and oxygen can be supplied. These organisms can include, but are not limited to Hydrogenobacter sp., Hydrogenobacter thermophilus, Hydrogenovibrio marinus, Helicobacter sp., Helicobacter pylon, Hydrogenophaga sp., Hydrogenomonas sp., Cupriavidus necator, Rhodococcus opacus, Alcaligenes sp., Alcaligenes eutrophus, Alcaligenes latus, Alcaligenes paradoxus, Alcaligenes ruhlandii, Aquaspirillum autotrophicum, Bacillus schlegelii, Pseudomonas carboxydovorans, Pseudomonas facilis, Pseudomonas fiava, Pseudomonas pseudofiava, Pseudomonas hydrogenovora, Pseudomonas hydrogenothermophila, Pseudomonas palleronii, Pseudomonas saccharophila, Pseudomonas thermophila, Seliberia carboxyhydrogena, Flavobacterium autothermophilum, Paracoccus denitrificans, Xanthobacter autotrophicus, X. autotrophicus, Arthrobacter sp. (1IX, RH 12), Mycobacterium gordonae, Nocardia autotrophica, and Nocardia opaca. Some contemplated organisms utilize hydrogen under anaerobic conditions, with sulfate or carbon dioxide as hydrogen acceptors (such as Desulfovibrio, Clostridium aceticum, Aceto-bacterium woodii, and Methanobacterium thermo-autotrophicum).

Yeast species which can be used in the invention include in particular Saccharomyces cerevisiae, Saccharomyces bayanus and Saccharomyces boulardii. Other suitable yeast species include Saccharomyces sp, Saccharomyces pastorianus, Saccharomyces carlsbergensis, Leucosporidium sp., Leucosporidium frigidum, Saccharomyces telluris, Candida sp., Rhodotorula sp., Trichosporon sp, Schizosaccharomyces pombe, Schizosaccharomyces sp., Sporidiobolus sp, Sporobolomyces sp., Candida tropicalis, group consisting of Xanthophyllomyces dendrorhous, Kluyveromyces lactis, Ogataea polymorpha, Metschnikowia fructicola, and any combination thereof. Of these, Saccharomyces sp, Leucosporidium sp. Rhodotorula sp., Trichosporon sp., Schizosaccharomyces sp., Sporidiobolus sp, Sporobolomyces sp., and Candida tropicalis are particularly contemplated.

Fungi which may be used in devices and methods of the invention include filamentous fungi such as Aspergillus japonicus, Aspergillus niger, Aspergillus foetidus, Aspergillus oryzfl Aureobasidium pullulans, Sclerotinia sclerotiorum and Scopulariopsis brevicaulis. Mould species include members of groups including Acremonium sp., Alternaria sp., Aspergillus sp., Cladosporium sp., Fusarium sp., Mucor sp., Penicillium sp., Rhizopus sp., Stachybotrys sp., Trichoderma sp., Trichoderma reese, Trichophyton sp., Aspergillus oryzae, Monascus purpureus, Penicillium sp., Penicillium nalgiovense, Fusarium venenatum, Geotrichum candidum, Neurospora sitophila, Rhizomucor miehei, Rhizopus oligosporus, Rhizopus oryzae, Geotrichum sp., Neurospora sp., Rhizomucor sp., Spinellus fusiger, and Spinellus sp. Of the moulds, the genera Acremonium sp., Alternaria sp., Aspergillus sp., Cladosporium sp., Fusarium sp., Mucor sp., Penicillium sp., Rhizopus sp., Stachybotrys sp., Trichoderma sp., and Trichophyton sp. are particularly contemplated.

Slime moulds refer to a number of groups of facultatively multicellular eukaryotes. Suitable examples for use in the present invention include Physarum polycephalum, Fuligo septica, Fuligo sp., Stemonitis furca, Stemonitis sp., Diachea leucopodia, Diachea sp., Trichia sp., Trichia varia, dictyostelids, Dictyostelium sp., Dictyostelium purpureum, Dictyostelium discoideum, myxomycetes, dictyostelids, and protosteloids, and in particular Acrasidis, Plasmodiophorids, Labyrinthulomycota, Fonticula, Nuclearia sp., Myxogastria, Stemonitis, and Physarum sp.

Microorganisms which are capable of photosynthesis may also be used in devices according to the invention. Possible organisms of this kind include members of groups such as Bracteococcus, Chlorella, Parachlorella, Prototheca, Pseudochlorella, and Scenedesmus. Other possibilities include Achnanthes orientalis, Agmenellum, Amphiprora hyalina, Amphora coffeiformis, Amphora coffeiformis linea, Amphora coffeiformis punctata, Amphora coffeiformis taylori, Amphora coffeiformis tenuis, Amphora delicatissima, Amphora delicatissima capitata, Amphora sp., Anabaena, Ankistrodesmus, Ankistrodesmus falcatus, Boekelovia hooglandii, Borodinella sp., Botryococcus braunii, Botryococcus sudeticus, Bracteococcus minor, Bracteococcus medionucleatus, Carteria, Chaetoceros gracilis, Chaetoceros muelleri, Chaetoceros muelleri subsalsum, Chaetoceros sp., Chlorella anitrata, Chlorella Antarctica, Chlorella aureoviridis, Chlorella candida, Chlorella capsulate, Chlorella desiccate, Chlorella ellipsoidea, Chlorella emersonii, Chlorellafusca, Chlorellafusca var. vacuolata, Chlorella glucotropha, Chlorella infusionum, Chlorella infusionum var. actophila, Chlorella infusionum var. auxenophila, Chlorella kessleri, Chlorella lobophora (strain SAG 37.88), Chlorella luteoviridis, Chlorella luteoviridis var. aureoviridis, Chlorella luteoviridis var. lutescens, Chlorella miniata, Chlorella minutissima, Chlorella mutabilis, Chlorella nocturna, Chlorella ovalis, Chlorella parva, Chlorella photophila, Chlorella pringsheimii, Chlorella protothecoides (including any of UTEX strains 1806, 411, 264, 256, 255, 250, 249, 31, 29, 25), Chlorella protothecoides var, acidicola, Chlorella regularis, Chlorella regularis var. minima, Chlorella regularis var. umbricata, Chlorella reisiglii, Chlorella saccharophila, Chlorella saccharophila var. ellipsoidea, Chlorella salina, Chlorella simplex, Chlorella sorokiniana, Chlorella sp., Chlorella sphaerica, Chlorella stigmatophora, Chlorella vanniellii, Chlorella vulgaris, Chlorella vulgarisf tertia, Chlorella vulgaris var. autotrophica, Chlorella vulgaris var. viridis, Chlorella vulgaris var. vulgaris, Chlorella vulgaris var. vulgarisf tertia, Chlorella vulgaris var. vulgarisf viridis, Chlorella xanthella, Chlorella zofingiensis, Chlorella trebouxioides, Chlorella vulgaris, Chlorococcum infusionum, Chlorococcum sp., Chlorogonium, Chroomonas sp., Chrysosphaera sp., Cricosphaera sp., Crypthecodinium cohnii, Cryptomonas sp., Cyclotella cryptica, Cyclotella meneghiniana, Cyclotella sp., Dunaliella sp., Dunaliella bardawil, Dunaliella bioculata, Dunaliella granulate, Dunaliella maritime, Dunaliella minuta, Dunaliella parva, Dunaliella peircei, Dunaliella primolecta, Dunaliella salina, Dunaliella terricola, Dunaliella tertiolecta, Dunaliella viridis, Eremosphaera viridis, Eremosphaera sp., Ellipsoidon sp., Euglena, Franceia sp., Fragilaria crotonensis, Fragilaria sp., Gleocapsa sp., Gloeothamnion sp., Hymenomonas sp., Haematococcus pluvialis, Haematococcus sp., Isochrysis aff galbana, Isochrysis galbana, Lepocinclis, Micractinium, Micractinium (UTEX LB 2614), Monoraphidium minutum, Monoraphidium sp., Nannochloris sp., Nannochloropsis salina, Nannochloropsis sp., Navicula acceptata, Navicula biskanterae, Navicula pseudotenelloides, Navicula pelliculosa, Navicula saprophila, Navicula sp., Nephrochloris sp., Nephroselmis sp., Nitschia communis, Nitzschia alexandrina, Nitzschia communis, Nitzschia dissipate, Nitzschia frustulum, Nitzschia hantzschiana, Nitzschia inconspicua, Nitzschia intermedia, Nitzschia microcephala, Nitzschia pusilla, Nitzschia pusilla elliptica, Nitzschia pusilla monoensis, Nitzschia quadrangular, Nitzschia sp., Ochromonas sp., Oocystis parva, Oocystis pusilla, Oocystis sp., Oscillatoria limnetica, Oscillatoria sp., Oscillatoria subbrevis, Parachlorella kessleri, Pascheria acidophila, Pavlova sp., Phagus, Phormidium sp., Platymonas sp., Pleurochrysis carterae, Pleurochrysis dentate, Pleurochrysis sp., Prototheca wickerhamii, Prototheca stagnora, Prototheca portoricensis, Prototheca moriformis, Prototheca zopfii, Pseudochlorella aquatica, Pyramimonas sp., Pyrobotrys, Rhodococcus opacus, Sarcinoid chrysophyte, Scenedesmus armatus, Schizochytrium, Spirogyra, Spirulina platensis, Stichococcus sp., Synechococcus sp., Tetraedron, Tetraselmis sp., Tetraselmis suecica, Thalassiosira weissflogii, and Viridiella fridericiana, Euglenophyceae, Prasinophyceae, Eustigmatophyceae, Bacillariophyceae, Prymnesiophyceae, Pinguiophyceae, Dinophyceae, Trebouxiophyceae, Bicosoecophyceae, Katablephariophyceae, Chlorophyceae, Haptophyceae, Raphidophyceae, Chysophyceae, Coscinodiscophyceae, Alveolata, Bangiophyceae, Rhodophyceae, Schizotrium sp., Crypthecodinium sp., Phaeodactylum sp. and Odontella sp., Odontella aurita, Botryococcus genus, Botryococcus sudeticus, Botryococcus braunii, Chlamydomonas sp., Chlamydomonas caudata, Chlamydomonas ehrenbergii, Chlamydomonas elegans, Chlamydomonas moewusii, Chlamydomonas nivalis, Chlamydomonas ovoidae, Chlamydomonas reinhardtii, Chlamydomonas mundane, Chlamydomonas dehoryana, Chlamydomonas cuiieus, Chlamydomonas noctigama, Chlamydomonas auiato, Chlamydomonas marvanii, Chlamydomonas proboscigera. In some embodiments, such organisms may be one or more of Haematococcus sp., Haematococcus pluvialis, Chlorella sp., Chlorella autotraphica, Chlorella vulgaris, Scenedesmus sp., Synechococcus sp., Synechococcus elongatus, Synechocystis sp., Arthrospira sp., Arthrospira platensis, Arthrospira maxima, Spirulina sp., Dysmorphococcus sp., Geitlerinema sp., Lyngbya sp., Chroococcidiopsis sp., Calothrix sp., Cyanothece sp., Oscillatoria sp., Gloeothece sp., Microcoleus sp., Microcystis sp., Nostoc sp., Nannochloropsis sp., Anabaena sp., Phaeodactylum sp., Phaeodactylum tricornutum, Dunaliella salina, some Arthrospira platensis, some Nannochloropsis sp. and Synechococcus marinus. In particular, Prototheca, Chlorella, Parachlorella, Pseudochlorella, Scenedesmus, Amphora sp., Anabaena, Chlorella aureoviridis, Chlorella vulgaris, Dunaliella sp., Dunaliella bardawil, Dunaliella salina, Euglena, Haematococcus pluvialis, Haematococcus sp., Nannochloropsis salina, Nannochloropsis sp., Nitschia communis Oscillatoria sp., Scenedesmus armatus, Schizochytrium, Spirogyra, Spirulina platensis, Stichococcus sp., Synechococcus sp., Tetraedron, Tetraselmis sp., Euglenophyceae, Odontella aurita, Botryococcus genus Chlamydomonas sp., and Chlamydomonas reinhardtii are contemplated.

Diatom species can include N. frigida, Nitzschia kerguelensis, N. lacuum, and in particular Phaeodactylum sp, Phaeodactylum tricornutum, Nitzschia sp., Cyclotella sp., and Cyclotella meneghiniana, and diatom classes like Bacillariophyceae, Coscinodiscophyceae, and Naviculales.

Rotifers, a group of microscopic and near microscopic animals, may also be used.

Capnophiles are also contemplated for use. These microorganisms thrive in the presence of high concentrations of carbon dioxide, and could particularly be used for applications where high carbon dioxide sequestration is desired.

Extremophiles refer to a number of groups of organisms which can tolerate unusual extremes in environment, typically high or low temperatures, extremes of pH, salinity, desiccation and/or radiation levels. Particularly contemplated examples which may be used in devices and methods according to the invention include members of the order Cyanidiales, Galdieriaceae, Cyanidioschyzon sp., Cyanidiophyceae class, Galdieria sp., Cyanidioschyzon merolae DBV201, Cyanidium daedalum, Cyanidium maximum, Cyanidium partitum, Cyanidium rumpens, Galdieria daedala, Galdieria maxima, Galdieria partita, and especially the species Galdieria sulphuraria, Cyanidium caldarium, and Cyanidioschyzon merolae.

Plant species, in particular aquatic plant species including green algae, may be cultured in devices and methods according to the invention. Whole plant organisms may be used where appropriate. Suitable species can include members of the duckweed family, Araceae, spotless watermeal, rootless duckweed, Lemnaceae, Lemna thalli, Lemna trisulca, Spirodela sp., Landoltia sp., Lemna gibba, Lemna minor, Lemna aequinoctialis, Lemna valdiviana, Lemna obscura, Spirodela polyrhiza, Wolffia arrhiza, Wolffia sp., and Spirodela sp. In particular, Lemnaceae, Wolffia arrhiza and Wolffia sp. are contemplated.

Plankton is a general term for ocean microfauna and microflora. Examples for use in the present invention include coccolithophores, dinoflagellates, metazoan plankton, and protozoan plankton, and in particular Emiliana sp. such as Emiliana huxleyi.

Amoeboids refer to various groups of cells or unicellular organisms which are able to change their shapes by the extension of pseudopods. Examples of organisms of this kind for use in the present invention include Chaos carolinense, Chaos diffluens, Chaos sp., Naegleria sp, Naegleria fowleri, Entamoeba sp., Cercozoan amoeboids, Euglypha sp., Euglypha rotunda, and Gromia sp., Gromia sphaerica, Foraminifera sp., Massisteria voersi, Massisteria sp., Pelomyxa palustris, Syringammina fragilissima, and Syringammina sp.

In addition, the invention may be used to culture cells from multicellular organisms. In particular, animal cells from animals such as livestock and poultry including chicken, duck, turkey; fish, bovine, or porcine cells, game or aquatic animal species, and insects, Particular cells which can be grown in devices and methods according to the invention include myocyte cells, adipocyte cells, epithelial cells, myoblasts, satellite cells, side population cells, muscle derived stem cells, mesenchymal stem cells, myogenic cells, myogenic pericytes, or mesoangioblasts. Myogenic cells here relate to cells from an embryonic stem cell line, induced pluripotent stern cell line, extraembryonic cell line, or somatic cells, modified to express one or more myogenic transcription factors. In particular, myocytes or similar cells may be grown for use in the production of so-called lab-grown meat, for the nutrition of humans or other animals. Totipotent cells deriving from human embryonic cells and human embryos are excluded.

Some organisms, whether native strains or genetically modified or engineered strains, can have the ability to uptake air-pollutants such as NO₂ (and other NOx such as NO, N₂O₂, N₂O₃, N₂O₅), SO₂ (and other SOx such as S₂O₂, SO, SO₃), VOCs, NH₃, or ‘greenhouse’ gases other than CO₂ such as N₂O. If so, these gases can be pumped in the gas chamber to then be transferred in the liquid media. These gases can also come from effluent gases.

In this respect, sulphur oxidizing organisms can also be grown in devices as described. These organisms carry out the oxidation of sulphur to produce energy. Some inorganic forms of reduced sulphur, mainly sulphide (H₂S/HS⁻) and elemental sulphur (S₈), can be oxidised by chemolithotrophic sulphur-oxidising prokaryotes, usually coupled to the reduction of oxygen (O₂) or nitrate (NO₃⁻). Most of these sulphur oxidisers are autotrophs that can use reduced sulphur species as electron donors for carbon dioxide (CO₂) fixation. This organisms could be grown using inside the chamber a gas mixture containing CO₂ and another, sulphur-containing gas to deliver the needed sulphur species into the liquid media, in particular where the membrane is permeable to such a gas. Alternatively the sulphur containing molecule could be added directly in the liquid media via nozzles, in either gasous or liquid (aqueous) form. Forms of sulphur which could be used either in the chamber (or by direct addition) include H₂S or using H₂S donor compounds such as NaHS or Na₂S. Relevant organisms include the Beggiatoaceae family, Thiobacilliaceae family, Sulfolobales order (Archaea), Sulfolobus genera, Acidianus genera, Hydrogenovibrio crunogenus, and the Desulfobulbaceae family. Relatedly, some Anaerobic sulfur oxidizing organisms can be photosynthetic autotrophs which obtain energy from sunlight but use reduced sulfur compounds instead of water as electron donors for photosynthesis.

In some embodiments, the organisms of the bioreactor are genetically modified to possess a specific trigger that is activated by exposure to a gaseous or vaporized stimulant that can be delivered into the atmosphere comprised within the chamber. When this stimulant is introduced into the chamber it diffuses across the membrane of the bioreactor and is delivered into the liquid media. The stimulant acts as a trigger and induces the organisms to react in a predetermined manner as intended by the genetic intervention. For example, the stimulant may induce the production or cease of production of a particular metabolite and/or may change the production rates of particular metabolites.

The above descriptions regarding the provision of O₂-enriched and/or CO₂ depleted atmosphere within the chamber is applicable to all other suitable gases, the control of which can be used for a variety of purposes.

Gases can be introduced into the chamber to control the pH of the liquid media comprised within the bioreactor. According to specific embodiments of the invention the concentration of CO₂ and/or ammonia (NH₃) within the atmosphere may be used to control the pH of the liquid media.

As described above, organisms may be modified (or may have a natural ability to) to respond to the presence or absence of certain gases by changing their physiological processes, and the gas mixture supplied to the atmosphere comprised within the chamber can be controlled to provide or remove such a gas.

The Chamber Atmosphere

The composition and/or quantity of the gas mixture supplied to the device may be controlled and moderated in response to a change in one or more parameters measured within the liquid media within the bioreactor, and/or in response to the metabolic or other physiological state of the cells comprised within the bioreactor. For example, parameter changes including a pH change in the liquid media could lead to the provision of a pH-affecting gas (like CO₂). Alternatively, the detection of a low O₂ concentration in the liquid media could lead to the supply of an increased level of O₂ in the input gas. Monitoring of the status of the liquid media and/or cells may be carried out through an auxiliary system controlling the device (see below).

Input gas may need to be pre-treated before its delivery to the gas-chamber, for example to remove substances which may be toxic to the cells or that may affect the cleanliness or transparency of the bioreactor or chamber surfaces. Pre-treatment of gaseous feed to the chamber may include any suitable technologies or strategies such as high efficiency particulate air (HEPA) filters and/or activated carbon filters, and can work to remove specific air pollutants, volatile organic compounds (VOCs), particulate matter of various grades (for example PM1, PM2,5, PM10), soot, and any other undesirable or otherwise toxic content.

According to a specific embodiment of the invention, a feed gas can be delivered in the chamber in the opposite direction of the overall direction of liquid media flow in the bioreactor. In this way a counterflow arrangement can be established wherein the feed gas with the highest O₂ concentration can be brought into contact with the liquid media with the lowest dissolved O₂ concentration (due to processes consuming O₂ occurring during liquid media flow through the bioreactor system), and likewise the gas with the lowest CO₂ concentration contacts the liquid media with the highest dissolved CO₂ concentration. This increases the concentration differential of the gases and so improves gas transfer efficiency. In another embodiment the feed gas with the highest CO₂ concentration can be brought into contact with the liquid media with the lowest dissolved CO₂ concentration (due to processes consuming CO₂ occurring during liquid media flow through the bioreactor system), and likewise the gas with the lowest O₂ concentration contacts the liquid media with the highest dissolved O₂ concentration.

Support Structures and Auxiliary Systems

The device can comprise a support structure that includes a frame, scaffold and/or manifold which serves to elevate and/or support the bioreactor within the chamber—as well as supporting a plurality of bioreactors within a chamber or a plurality of chambers where an array is comprised within the device. The support structure may also or alternatively maintain the shape and structure of the chamber itself, and/or in terms of directing flow of the gaseous atmosphere around the bioreactor comprised within the chamber. Additionally or alternatively, the support structure may further aid in the attachment of the device to a mount or other surface, and in providing stability of the device as a whole.

In a specific embodiment of the invention a support structure can be comprised of an extrusion of a rigid solid material, and is preferably lightweight, as described in the exemplary device below. The support structure has no need to be transparent, even in embodiments where part or all of the chamber walls are transparent, although it can be, and may be manufactured from any suitable material, which is typically a strong, light and non-toxic material, with high resistance to oxidation, corrosion, extremes of temperature and ultraviolet radiation. The support structure can comprise a substantially solid material, or can comprise a porous structure to decrease its weight while maintaining strength.

In particular, it is contemplated that support structures may be used to support the bioreactors themselves, in order to help them bear the weight of the liquid media and cells that are comprised within them. In particular towards the middle of a section of a bioreactor, the weight of the contents may cause sagging, stretching or weakness of the material comprising the bioreactor. In addition, blockage or excessive pressure of the liquid media within the bioreactors may cause swelling, which could lead to costly and inconvenient damage or breakage of the membranes which comprise the bioreactors. Therefore, one or more bioreactor support structures, or support assemblies, contacting the underside of the bioreactors may be used.

Such bioreactor support structures may comprise fins, gutters or cradles in which the bioreactors lie, which may be protrusions of the lower internal wall and/or any other internal wall of the chamber. The bioreactor support structures may be a net, or a series of cords, strings or cables attached to the side internal walls of the chamber, and/or to any other internal wall of the chamber. The bioreactor support structures may advantageously be discontinuous, that is, comprising gaps, to enable gas from the chamber atmosphere to contact the membranes of the bioreactor. Suitably, the bioreactor support structures may be a flexible, or typically a rigid or semi-rigid mesh, which has a plurality of perforations or holes, which can support the bioreactor while still allowing gas to access the membrane of the bioreactor for effective gas exchange, even where it contacts the support structure. Indeed, it is contemplated that in some arrangements not only the underside of the bioreactors may be contacted by the bioreactor support structures, but the sides and tops may also be contacted. This may also aid in preventing swelling (radial expansion) of the bioreactors and thereby protect against bursting. In some embodiments, a bioreactor support structure comprises a flexible, semi-rigid or rigid mesh which substantially surrounds the cross-sectional circumference of at least part of the bioreactor. In other embodiments, the mesh surrounds the entire cross-sectional circumference of the bioreactor to prevent swelling (radial expansion) of the bioreactor and thereby protecting against rupture, and to control the cross-sectional shape of the bioreactor (for example controlling the diameter when the bioreactor is in a tubular form). The mesh may enclose all or a part of the elongate bioreactor. The density of the holes or apertures within the mesh may vary depending on position and the need for support. For example, the mesh around the underside of the bioreactor may have smaller, fewer, and/or more widely spaced holes to provide more support, while the mesh around the top of the bioreactor may have larger, more numerous, and/or more closely spaced holes to aid in gas access to the bioreactor. The mesh can be made in any suitable way, it may be made of connected strands, strings, wires or cables; it may be made of sheet material with holes or other perforations, or from a woven or knitted fabric. The mesh can be of any suitable material, for example a plastic polymer, typically a plastic polymer containing UV stabilizers. The mesh can be of any suitable thickness, it may be not less than 0.1 mm and not more than 3 mm thick, typically bellow 1 mm thick. The holes of the mesh can be of any shape and dimensions, they may be not less than 0.1 mm and not more than 10 cm wide, suitably not more than 10 mm, not more than 5 mm, typically not more than 3 mm.

These supports may also advantageously allow the bioreactors to be suspended above the lower internal wall of the chamber, which can allow gas from the chamber atmosphere to access parts of the bioreactor membranes other than those exposed at the top, and can also allow for vertical arrangements (or ‘stacks’) of multiple bioreactors to be arranged in the same chamber. Suitably, the support assemblies may be arranged as a series of shelves or armatures which are arranged to support a three-dimensional array of bioreactors. The shelves, which may be any support structure discussed, can be arranged in a horizontal and/or vertical; parallel and/or anti-parallel array.

Support structures may also be present on the inside of the bioreactors to provide support or maintain the shape of the bioreactors, or may be comprised within the membranes of the bioreactors themselves. In particular, the membranes may be composite materials comprising an internal film, mesh, ribs or other structures to help the bioreactor maintain shape and strength, while preserving sufficient gas permeability. Such composites could be produced with co-extrusion manufactory techniques.

Suitably, the support structure can comprise plastics, such as bioplastics, thermoplastics, thermosetting polymers, amorphous plastics, crystalline plastics, synthetic polymers such as acrylics, polycarbonates, polyesters, polyurethanes carbon fibre composites, Kevlar composites, carbon fibre and Kevlar composites or fibre glass; metals or metal alloys such as steel, mild steel, stainless steel, aluminium or titanium; natural materials such as wood or coated wood; or carbon-based materials such as graphene, carbon nanotubes or graphite.

The bioreactors of the device may be connected to an auxiliary system which controls the supply and condition of the gas and/or liquid media used. Depending on the application of the device, the auxiliary system can be of any degree of complexity and composed by any kind of auxiliary components.

In a suitable embodiment of this invention, the device is connected to an auxiliary system mainly composed by conduits for gas and for liquid media, water tanks, gas tanks or canisters, pumps for gas and liquid media, valves, biomass-separators, artificial lighting systems (especially if natural light is not present), water temperature control systems, sensors and computers. One component, a plurality of components or all of the components of the auxiliary system can be provided inside or outside the chamber. The different features of the auxiliary system do not have to be all comprised together, but may be dispersed in different parts of the system as a whole. For example, biomass separators, gas outlets and/or inlets for nutrients may be included in connectors between individual bioreactors.

The conduits and reservoirs (water tanks) can be of any type and of any suitable material.

The pumps can also be of any type; typically the liquid pumps are peristaltic pumps which can reduce the contamination risk of the liquid media and the breakage of the cells used due to the peristaltic tube being the only component in contact with the liquid media. In some embodiments diaphragm pumps (also known as membrane pumps) can be used. Diaphragm pumps create relatively little friction with the liquid media and so can have advantages in the reduction of cell breakage and the risk of contamination. In some other embodiments screw pumps, progressive cavity pumps and gear pumps can be used. Progressive cavity pumps create relatively little friction with the liquid media and so can have advantages in the reduction of cell breakage while being able to pump liquid at high flow rates.

Biomass-separators can be of any type known to the skilled person; suitably the biomass-separator is a centrifuge type bio-separator, a filtering system comprising small-aperture meshes, a sieve, and/or microfiltration/nanofiltration devices, and/or a sedimentation device, and/or clarification process. Multiple biomass-separation devices can be installed in series, for example an initial clarification process or microfiltration device followed by a centrifuge.

The liquid media temperature control can be of any type known to the skilled person; typically, the liquid media temperature is controlled by controlling the temperature of the gaseous atmosphere within the chamber. The temperature of the gaseous atmosphere within the chamber can be heated and/or cooled by any suitable component; typically, it is cooled by an air conditioning unit within the chamber or connected to the chamber through an inlet and an outlet. In other embodiments, the liquid media temperature controls comprises a heating or cooling component which may be suitably installed around or inside parts of the conduits, around the bioreactor sections, before the gas-inlet of the chamber and/or around or inside the reservoir. Infrared light transmission onto transparent or semi-transparent conduits can also be a way to heat liquid media. The heating components can be of any type, and suitably can comprise heat-exchange mechanisms. Excess heat from the liquid media generated by physiological processes or high environmental temperatures may be used to heat water for domestic or industrial purposes, or water from sources such as drain water, storm water, sewage water and/or grey water may be used to remove excess heat. Likewise, liquid media may be heated or cooled when necessary using heat or cold generated from domestic or industrial sources. In some embodiments the heat may be generated by electric heaters that converts an electric current into heat. In some other embodiments heating and/or cooling components can be heat exchange devices of any suitable type, such as heat exchangers between liquid and gas, heat exchangers between two liquids, heat exchangers between two gasses, air conditioning units (AC), double pipe heat exchangers, or plate heat exchangers. The air conditioning of the atmosphere within the chamber is suitably carried out within the chamber or in the location of the auxiliary system, before the gaseous mixture arrives in the chamber. Heat exchange between two liquids is suitably carried out in the location of the auxiliary system, before the liquid media arrives in the bioreactors.

An artificial lighting system can be used that comprises any artificial light source types known to the skilled person, suitably the lighting system comprises LEDs, typically the artificial light source is designed and/or controlled to emit specific wavelengths of electromagnetic radiation (light) corresponding to the photosynthetically active radiation (PAR) needs of any phototrophic microorganisms contained within the device and/or to promote specific biological activity, thereby increasing the production of specific products in the biomass, for example by using LEDs that emit specific wavelengths. For example an LED-based light source can emit wavelengths between approximately 620 nm and 750 nm (red light) to promote the production in some organisms of pigments that absorb mostly red light, such as the pigment phycocyanin. Artificial lighting systems may be comprised within the support structure that comprises arrays or strips of LEDs or optic fibres. The intensity and quality of the light emitted by the lighting systems could be controlled automatically (following inputs from any kind of sensors like PAR sensors, humidity sensors, temperature sensors, chemical sensors, pH sensors and so on) to promote specific microbial physiological activities and/or to respond to environmental changes and/or to increase or modify the biomass production. Similarly the amount of light transmission (either being natural or artificial light) through a ‘switchable’ or ‘smart glass’ material as discussed above can be automatically controlled for similar reasons.

In some embodiments an artificial lighting system may provide wavelengths of light which can be used to sterilise or disinfect part or all of the bioreactors and/or chambers of the invention. This can be as, or in addition to, a cleaning, disinfection or sterilisation process as discussed below. In particular such lighting systems may produce ultraviolet (UV) radiation which can kill or damage bacteria and other unwanted contaminant organisms. Suitably, the UV radiation is short-wavelength UV, sometimes called UVC. The source of the UV radiation in such systems may typically be a UV lamp, suitably a UV-producing LED. The wavelength of the UV radiation may comprise wavelengths between 260 and 270 nm. Suitably, wavelengths below about 254 nm may be excluded or blocked to reduce the production of ozone. In some applications, ozone production may be desired, for its additional disinfectant properties, and the wavelength of the UV radiation may be chosen to encourage this.

Since UV radiation can be harmful to humans, in particular to skin and eyes, such UV disinfection systems can suitably be used in embodiments where the walls of the chamber are substantially opaque or impermeable at least to the UV wavelengths used. Alternatively, the chamber can be covered or coated with such an opaque or UV-impermeable layer before activation of the UV disinfection system. Additionally, since UV radiation can age or damage many types of material, such as several polymers, any vulnerable materials (which may include the bioreactors) may be removed from the chamber before activation of the UV system, or the system or device may be arranged in such a way as to shield the vulnerable materials from the UV radiation.

According to one specific embodiment of the invention, when the biomass concentration in the liquid media comprised within the bioreactor reaches the desired level, a 3-way valve directs the flow into a biomass-separator which separates at least a part of the biomass from the liquid media, the isolated biomass proceeds into a receptacle for additional processing, while the liquid media is directed back into the reservoir. It may be necessary to regenerate the liquid media before returning it to the bioreactors. In some cases the liquid media will contain metabolites produced by the cultured organisms; these metabolites may need to be destroyed to maintain optimum growth rates, as in many cases the excessive presence of such metabolites causes a reduction in growth. Such metabolites can be removed utilising filtration systems, UV treatment and/or chemical treatments. Alternatively the liquid media filtered from the biomass separation process can be discarded. This action of directing the flow into the biomass-separator can be performed periodically and for a predetermined period of time before the valve changes the flow path into the reservoir again. This timing can be optimised with respect to each application, the microorganism used, the surrounding environment and physical location of the device. In another embodiment instead of a binary switch, the valve can change the aperture of the channel thereby controlling the flow rate and amount of liquid media that is delivered to the biomass separation process.

Nutrients can be periodically introduced in the system directly into the reservoir. Water and/or microorganisms in liquid media, or cleaning fluid, can be similarly introduced.

All sorts of other system components can be utilised, as example a controllable pressure valve or pressure regulator can be placed in the system, in this example the pressure valve can control the volumetric change of the unit through the effects of changes in the liquid or gas pressure. Some valves can control the flow rate into the units.

Supplementary air and/or air enriched with O₂ and/or other gases can optionally be introduced in the main bioreactor supply conduit if required. Vents can be installed in the conduits to remove gas that has accidentally entered the hydraulic system, for example during installation of the system, and are typically located in the highest location of the system to facilitate the expulsion of undesirable gas.

Sensors comprising transparent/translucent electrically conductive materials and/or any other electrically conductive materials can be provided on any surface of the chamber (inside or outside the chamber) to monitor conditions such as irradiance levels, temperature, humidity or other environmental conditions. These sensors or similar sensors, if located inside the chambers may be used to detect gas concentration levels, humidity and/or temperature in the chamber.

Embodiments and/or the auxiliary system of the invention can include embedded sensors which can be used, for example, to monitor chemical concentrations such as CO₂ concentrations and/or O₂ concentrations in liquid media and/or atmosphere; and/or to monitor temperature and other environmental and biological parameters, such as toxicity levels and/or to monitor the biomass concentration and/or the total cell density and/or the viable cell density and/or the activity of the microorganisms in the liquid media.

Sensors can be embedded entirely or partially in the bioreactor or the chamber, in the auxiliary system(s) of the tanks or conduit, and/or in control or support structures and/or be attached to the inside or outside of external layers or on surface of internal additional components.

Sensors can permit the monitoring of the environment inside the bioreactor of the device, in order to enable control of parameters including, but not limited to, liquid media flow rate, liquid media quality, nutrient levels, temperature, biomass extraction rate, gas mixture, gas flow rate, gas chamber pressure, and lighting intensity (and/or optical shielding such as provided by ‘smart glass’). The purpose of this control is to optimise the metabolic efficiency of the cells contained within the device, and/or to stimulate specific metabolic/microbial activities and hence to optimise the efficiency of generation of biomass and/or modify its composition.

Similarly, sensors can permit the monitoring of the environment inside the chamber of the device, in order to enable control of parameters including, but not limited to, gas flow rate, quality, composition, temperature, optical clarity and humidity.

Cleaning and Sterilisation

A cleaning procedure can be actuated to clean and/or sterilise bioreactor units and/or the conduits and/or the water tank and/or all the auxiliary systems and/or the chamber. Cleaning takes place when it is necessary to flush the system through, to collect all biomass in the system, or for temporary shutdowns. A “cleaning fluid” can be made of any compound known to the skilled person. It may comprise hydrogen peroxide, ethanol, water, saltwater, detergents, bleach, surfactants, alkali, it may be CIP100 or CIP150 from Steris or any other suitable cleaning composition. The cleaning fluid can enter the system through specific conduits (inlets) in any point of the system and can exit at any point of the system (outlets) to permit cleaning in specific locations only, if desired, instead of cleaning the entire system. Typically, a cleaning liquid like CIP100 is heated to desired temperature, typically over 30° C., and a turbulent flow is maintained for a determined period of time. The cleaning fluid may also be gaseous in nature and can comprise steam, heated air or water vapour, suitably supplied at temperatures above 120° C.

A sterilisation procedure aims to destroy and remove any and all organisms within the system, for permanent shutdown, decontamination. This approach may include pumping fluid into the system, for example steam or a low-temperature dry vapour of hydrogen peroxide. Sterilisation may also comprise the use of electromagnetic radiation, typically UV radiation, to disinfect any of the components of the invention, as discussed above. An advantage of a hydrogen peroxide dry vapour is that it does not require high pressure for effective sterilisation. Where it is necessary to pressurise a sterilisation fluid such as steam for effective sterilisation, it may be advisable to first pressurise the chamber atmosphere and subsequently the inside of the bioreactors, in order to avoid damage or bursting of the bioreactors.

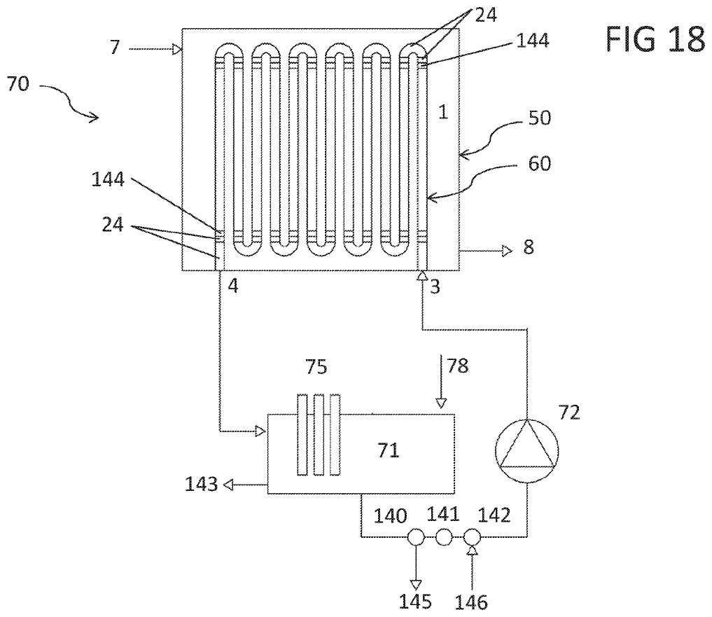

In some embodiments (as shown in FIG. 18) a series of valves (140, 141, 142), a discharge outlet (145) and an auxiliary inlet (146) may be used during the cleaning, sterilization, start-up, inoculation, liquid media removal, biomass harvesting, and/or growth media introduction procedures of the system. For example to replenish a soiled cleaning liquid previously used to clean the bioreactors with a new sterilising solution, the central valve (141) will be closed, the other two valves (140, 142) will be open and the pump (72) will continue to run to allow the soiled cleaning liquid to be discharged from the discharge outlet (145) and to allow the new fresh sterilising solution to be introduced in the system from the auxiliary inlet (146).

Biomass Collection

An advantage of some embodiments of the invention is that biomass can be generated continuously within the unit and can be harvested on a continuous basis.

The biomass which can be collected from some embodiments of the invention varies depending on the setup and condition of the devices of the invention, the cells comprised within the bioreactors, the desires of the users of the invention, and the nature of the separation and treatment of the biomass. The general types of biomass which can be collected from the invention in various embodiments can include, but is not limited to: metabolic products of the cells; secreted proteins and other cellular products; products of photosynthesis, aerobic respiration and/or anaerobic respiration; cell contents including cell organelles, cell membranes, cell walls; macromolecules including polysaccharides such as starches and cellulose, fats, phospholipids, proteins, glycoproteins, glycolipids and/or nucleic acids; carbohydrates such as monosaccharides, disaccharides and/or oligosaccharides; fatty acids and/or glycerol; whole organisms including cells, agglomerations and/or colonies of unicellular organisms or whole multicellular organisms or parts thereof.

The applications of biomass produced by embodiments of the invention can include food; feeds for animals, plants or any organisms; feeds suitable for aquatic use such as for aquatic animals or other organisms; pharmaceuticals; cosmetics; fuels; biochemical; oils; substitutes for mineral oils and mineral oil products; manufactory oils; and vaccines.

Biomass accumulates in the liquid media within the bioreactors. The biomass can be harvested directly from the liquid media. Biomass is mostly formed in the system during travel of the liquid media through the bioreactors, as this is where it spends most time, and is supplied with O₂. In order to release biomass, liquid media enters the device via the one or more inlets, passes through the one or more channels and exits the device, together with biomass that is carried in the flow, via the one or more outlets. The outlet can be connected to a suitable receptacle for receiving the harvested biomass.

A particular advantage of the present invention is the ability for products to be harvested on a continuous, semicontinuous or batch basis, due to the ability to continually circulate the liquid media through the system. Harvest can occur for example when a particular cell density is reached, which can be expressed in grams per litre, such as at least about 1 g/l, at least about 2 g/l, about 5 g/l, about 10 g/l, about 20 g/l, about 30 g/l, about 50 g/l, about 75 g/l, or at least about 100 g/l. For example, if a percentage of the liquid media passing through the auxiliary system after flowing through the bioreactors is constantly harvested, and liquid media is added to the system to replace it, a continuous harvest can be attained. Depending on the organism cultured, the volume of the bioreactor system, and the time taken for liquid media to flow through the entire system, any suitable amount can be harvested. For example 100% of the liquid media can be harvested by the auxiliary system, or the harvest can take no more than 90%, no more than 70%, 50%, 30%, 20%, 10%, 5%, 1%, or no more than 0.5% of the liquid media when it flows out of the bioreactors.

Alternatively, biomass can be harvested intermittently, on a semicontinuous basis. For example, a percentage of the biomass can be harvested from the device of the invention frequently, on an hourly, daily or weekly basis. For instance, harvests may take place weekly, daily, every 12, 6, 4, or 2 hours, or every hour. The harvested volume can be replaced by the addition of liquid media (with or without additional organisms), and additional nutrients. Harvest can be regular, after a set period of time, or can be triggered by reaching a certain organism density or biomass concentration or intended product concentration. As above, the amount taken can vary appropriately, based on the organism and the system. For example the harvest during semicontinuous operation can take no more than 98%, no more than 95%, 90%, 70%, 50%, 30%, 20%, 10%, 5%, 1%, or no more than 0.5% of the liquid media when it flows out of the bioreactors.

Such continuous or semi-continuous methods have the benefit of a predictable and continual production of biomass, do not require new or additional organisms to be introduced into the bioreactor after harvesting, and can allow for reduced variability in product, in contrast to batch processes which are more common with standard fermenters. In a fermenter setup, the risk of contamination means that continuous processes are rarely suitable.

A batch process can however also be used, and would involve harvesting the entire volume of liquid media at one time after a set time has elapsed, or a set density of organisms or biomass or product has been reached. This can involve draining the entire system and/or flushing it through with replacement fluid. This approach can be used in conjunction with any continuous or semi-continuous methods, for example when it is required to clean the system or replace the cultured organisms.

In some embodiments (as shown in FIG. 18) a series of valves (140, 141, 142), a discharge outlet (145) and an auxiliary inlet (146) may be used during the cleaning, sterilization, start-up, inoculation, liquid media removal, biomass harvesting, and/or growth media introduction procedures of the system. For example is to replenish growth media consumed by the organisms and to remove liquid media from the system at the same time, the central valve (141) will be closed, the other two valves (140, 142) will be open and the pump (72) will continue to run to allow the liquid media to be discharged from the discharge outlet (145) and to allow the new liquid media with growth media to be introduced in the system from the auxiliary inlet (146).

Applications

The device of this invention can be utilised for many applications, primarily biomass production, but also carbon dioxide production, the sequestration of nitrogen oxides or other gases, or where the removal of pollutants is needed, or where waste water treatment is needed, or even for aesthetic or decorative applications such as urban furniture or functional artistic installations. The device can thereby be used at locations such as warehouses, breweries, industrial buildings and the like. Similarly, the device can be used in conjunction with transportation vehicles, such as ships, aeroplanes, cars, trucks and other road vehicles. The device can be used indoors and/or outdoors. In some embodiments, the devices of the invention can provide carbon dioxide for devices which aim to supply increased carbon dioxide to support the growth of photoautotrophic organisms, for example gas-permeable membrane bioreactors as described in WO2017/093744 and WO2018/100400.

Suitable applications for the device of this invention can be any indoor and/or outdoor architectural applications including, but not limited to, being part of a building façade, roofs, sun-canopies, sun shades, windows, and/or indoor ceilings, indoor walls, or indoor floors. Thermal insulation can also be provided to these buildings by the invention.

Additional suitable applications for the device of this invention can be intensive biomass production applications, including, but not limited to, outdoor intensive biomass production plants using mostly natural light sources, indoor intensive biomass production plants, such as in greenhouses. The biomass can contain food ingredients and/or additives and/or can be used as a protein source for human or animal consumption, or for plant or other fertilising purposes. Further suitable applications for the device of this invention can be together with infrastructures, including, but not limited to, urban infrastructures, motorways, bridges, industrial infrastructures, cooling towers, highways, underground infrastructures, traffic sound barriers, silos, water towers, or hangars.

### FIGURES

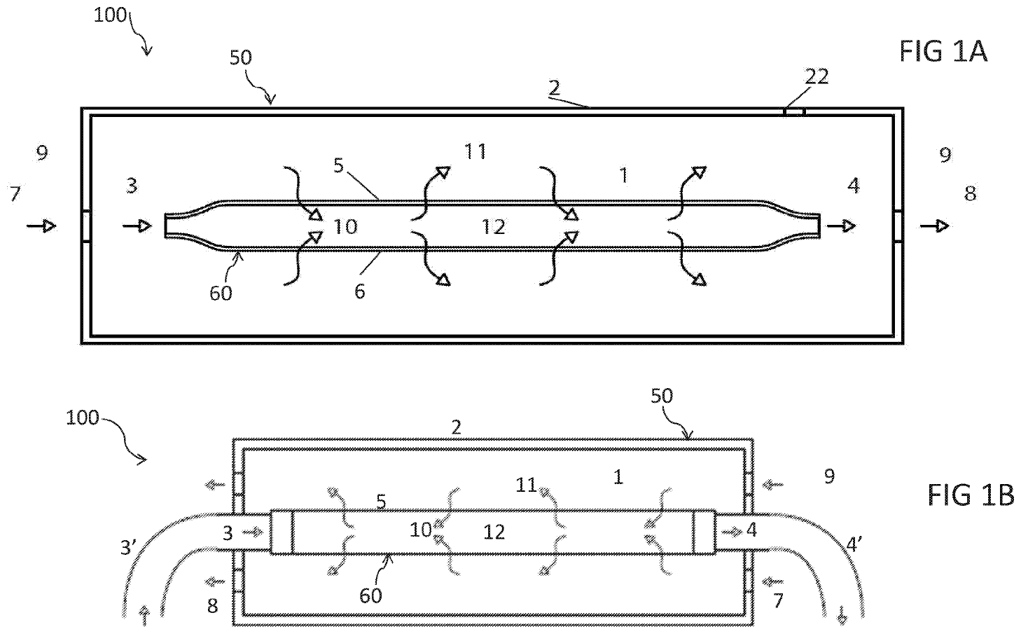

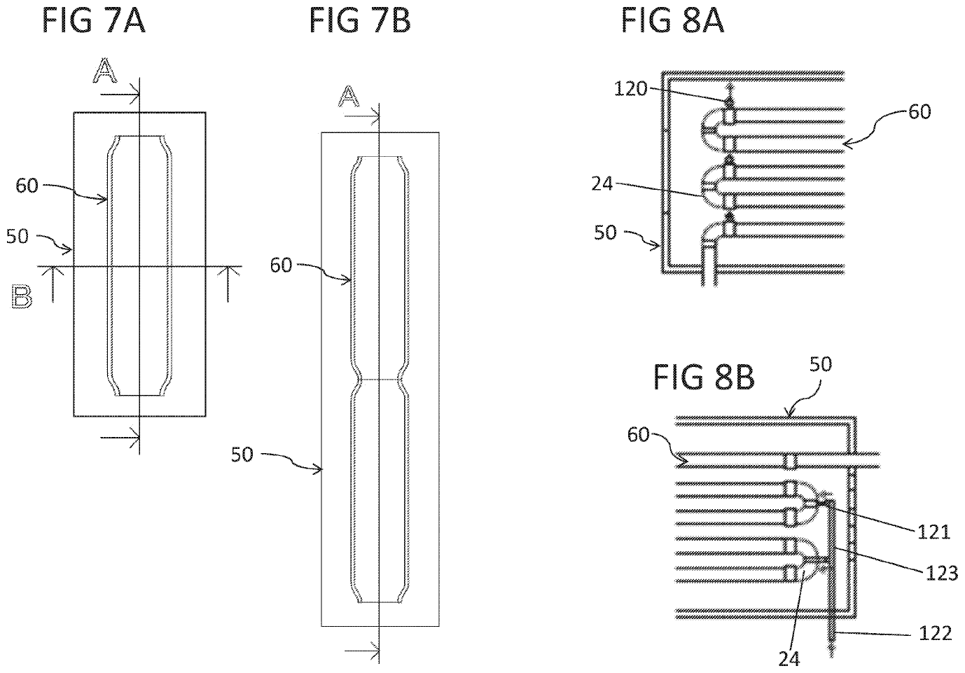

FIG. 1A is a diagram showing a cross-section (see Section A of FIG. 7a) of a device according to an embodiment of the invention (100), comprising a linear bioreactor (60) comprising at least one inlet (3) and outlet (4) located on opposite sides, and at least one outer layer (5, 6), part or all of which is permeable to gases, and liquid media comprising at least one cell (12) contained within the bioreactor. The bioreactor is surrounded on substantially all sides by an atmosphere (1) defined by its enclosure within a chamber (50) which comprises walls (2), an inlet (7) and an outlet (8). The chamber (50) and chamber walls (2) separate the atmosphere (1) from the outside atmosphere (9). In some embodiments the chamber further comprises a chamber valve (22) for the removal of gas from the atmosphere (1). The potential transfer of gases (10) is shown from the atmosphere (1) to the bioreactor contents (12) and also (11) from the bioreactor contents to the atmosphere (1).

FIG. 1B is a drawing of a similar device, where the inlets and outlets of the bioreactor are connectors which may be clamped to the bioreactor. The bioreactor is in a tube shape. Liquid media is supplied to the bioreactor though piping (3′, 4′), for example from an auxiliary system. The air inlet (7) introduces atmospheric air which has been cooled or heated as appropriate, and filtered. In this arrangement, oxygen is shown passing into the bioreactor, and carbon dioxide and water vapour passes out.

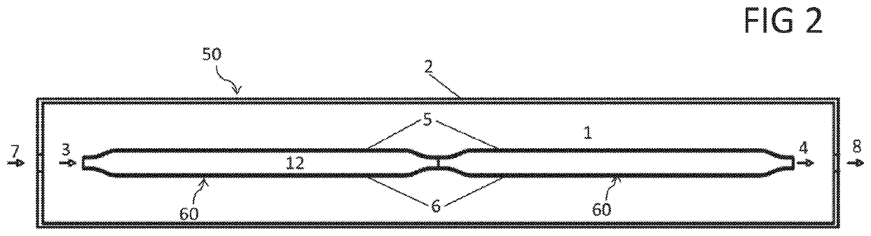

FIG. 2 shows a cross-section (see Section A of FIG. 7B) of an arrangement according to another embodiment of the invention wherein two bioreactors (60) are directly connected in series such that their liquid media (12) is in fluid communication, and the bioreactors are contained within a single chamber (50). In some embodiments more bioreactors may be connected within a single chamber.

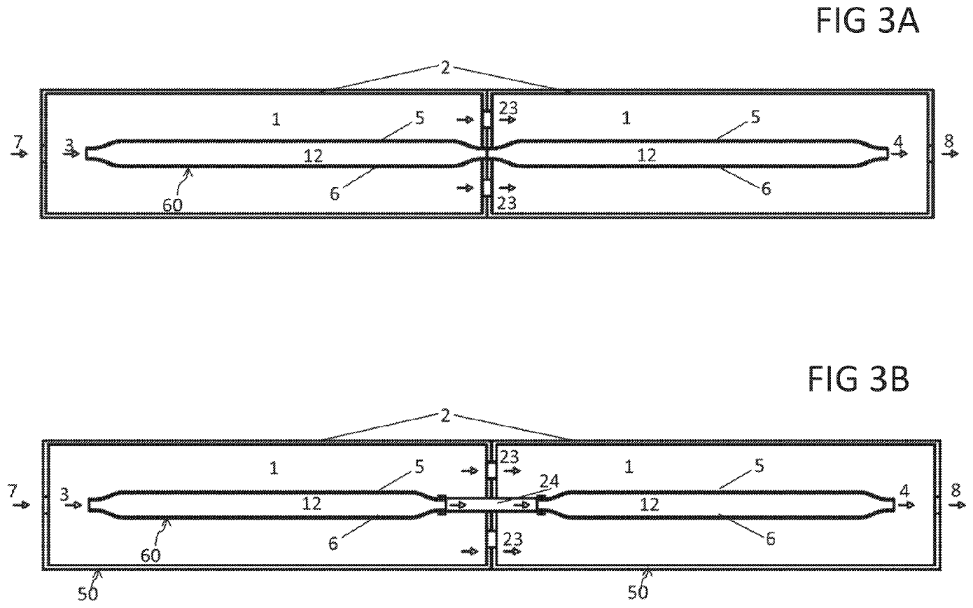

FIGS. 3a and 3b show cross sections of an arrangement according to another embodiment of the invention wherein two bioreactors (60) are directly connected in series, wherein each bioreactor (60) is contained within a chamber (50). The atmospheres (1) of the chambers (50) are in fluid communication with each other through apertures (23) in the chamber walls (2). The bioreactors may be connected via a conduit (24).

FIG. 4 shows a cross section of an arrangement according to another embodiment of the invention where five pairs of bioreactors are connected in series, with every successive pair arranged to run in an antiparallel direction from the previous pairs. The bioreactors are connected by connectors or conduits (24), which can simply connect one member of a bioreactor pair to the next, or can connect two pairs by using a curved connector or conduit, allowing for the antiparallel flow directions to be set up. Some or all of these connectors can contain valves (29), which may be automatic, and may for example be solenoid or diaphragm valves, to prevent flow of liquid media when desired.

FIG. 5 shows a cross section of an arrangement according to another embodiment of the invention where five pairs of bioreactors (60) are connected in parallel. The piping supplying and retrieving liquid media to and from the bioreactors splits and is connected to the ends of the bioreactors with connectors. The views shown in FIGS. 4 and 5 can be cross-sections taken either horizontally or vertically, that is, the multiple bioreactor pairs can respectively be arranged one next to another in a horizontal plane, or arranged one on top of another, in a vertical plane.

FIGS. 6A and 6B show perspective views of arrangements of bioreactors which may be used in some embodiments of the invention. The bioreactors in 6A are arranged in series, with bioreactors arranged in pairs, with each successive pair arranged to run in an antiparallel direction from the previous pair. Multiple layers are used, such that the bioreactors are arranged in three-dimensional space. In FIG. 6B, the flow path is split into 5 parallel streams, which flow into different bioreactor pairs. These flow paths however also comprise multiple pairs of bioreactors arranged in series, again with each successive pair arranged to run in an antiparallel direction from the previous pair.

FIG. 6C shows another perspective view of a three-dimensional array of bioreactors, which can be connected in any suitable way.

FIG. 6D shows a cross-section of a three-dimensional array of bioreactors (60) comprised within a chamber (50), with the distances marked between neighbouring bioreactors horizontally (110) and vertically (111), the width (112) and height (113) of the bioreactor array, and between the outermost part of the bioreactor array and the chamber itself (114).

FIGS. 7A and 7B show planar sections A and B through representations of the device according to some embodiments of the invention,

FIGS. 8A and 8B show additional optional features which may be comprised within any and all connectors or conduits of systems according to some embodiments of the invention. FIG. 8A shows that the conduits (24) may have one or more vents (124) which may be used to remove any unwanted gas within the bioreactor systems. Vents may also be used to allow gas to enter the bioreactors, for example during maintenance or during draining of all or part of the system. FIG. 8B shows that the conduits may have one or more inlets (121) for the introduction of a continual or intermittent supply of glucose, nutrients and/or any other kind of liquid or gaseous mixture. The inlet can be supplied through a supply line (123) from a source (122) which may originate outside the chamber (50).

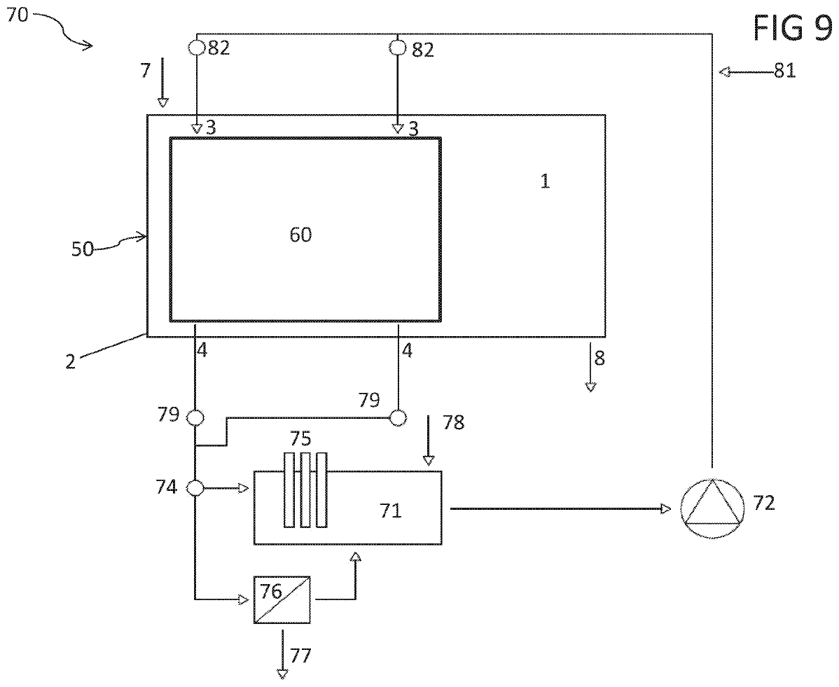

FIG. 9 shows a suitable system (70) of one embodiment of the invention, comprising any embodiment of one or more bioreactors according to the invention (60) as described herein, within one or more chambers (50). The liquid media (12) comprising cells in a reservoir (71) is conveyed by a pump (72) into a bioreactor through the inlet (3). The one or more bioreactors (60) are enclosed within a chamber (50) which also encloses an atmosphere (1), controlled by gas movement through an inlet (7) and outlet (8). The liquid media passes through the one or more bioreactors, while gas transfer between the liquid media in the bioreactor(s) and the atmosphere (1) occurs through the membrane layers of the unit substantially as shown, for example, in FIG. 1A. The liquid leaves the unit through the outlet (4) and reaches a 3-way valve (74) which directs the liquid media back into the reservoir (71), closing the circuit. Sensors (75) in the reservoir (71) measure the values of the culturing parameters and send outputs to the computers which then control operations of the auxiliary system's components, such as pumps, valves, artificial light systems (if used), temperature control systems, and biomass-separators. Computers also control supply of gases to the chamber atmosphere (1) through the inlet (7) and gas removal through the outlet (8).

When the biomass concentration in the liquid media reaches the desired level, the 3-way valve (74) directs the flow into the biomass-separator system (76) which separates the biomass from part of the liquid media, the isolated biomass proceeds into a receptacle (77) for additional processing, while the liquid media is directed back into the reservoir (71). This action of directing the flow into the biomass-separator can be performed periodically and for a predetermined period of time before the valve (74) changes the flow path into the reservoir (71) again. This timing can be optimised with respect to each application, the microorganism used, the surrounding environment and location of the device. Alternatively the 3-way valve (74) can regulate the flow to the reservoir (71) and the biomass separation system (76) to enable a continuous harvest of biomass while allowing for dynamic control of the quantity of biomass removed from the system at a given time. For example the valve (74) can deliver between 0% and 100% of all the liquid media that pass through the valve to the biomass separation system (76).

Nutrients can be periodically inserted (78) in the system directly into the reservoir (71). Water and/or cells in liquid media, or cleaning fluid, can be similarly introduced.

All sorts of other system components can be utilised, as example a controllable pressure valve or pressure regulator (79) can be placed in the system, in this example the pressure valve can control the volumetric change of the unit through the effects of changes in the liquid pressure. Some valves (82) can control the flow rate into the units.

Supplementary air and/or air enriched with oxygen and/or other gases can optionally be introduced (81) in the main conduit if required, in addition to the gas supply to the chamber. Vents can be installed in the conduits to remove gas that can accidentally enters the hydraulic system, for example during installation of the system, and are typically located in the highest location of the system to facilitate the expulsion of undesirable gas.

A cleaning procedure can be actuated to clean and/or sterilise the unit and/or the conduits and/or the water tank and/or all the auxiliary system and/or the gas chamber. The cleaning procedure can be performed by using steam or heated air or water vapour as a cleaning medium. A “cleaning fluid” can be made of any compound known to the skilled person. It may comprise ethanol, water, hydrogen peroxide (H₂O₂), salty water, detergents, bleach, surfactants, alkali or any other suitable cleaning composition. The cleaning liquid can enter the system through specific conduits in any point of the system and can exit at any point of the system to permit cleaning in specific locations only, if desired, instead of cleaning the entire system.

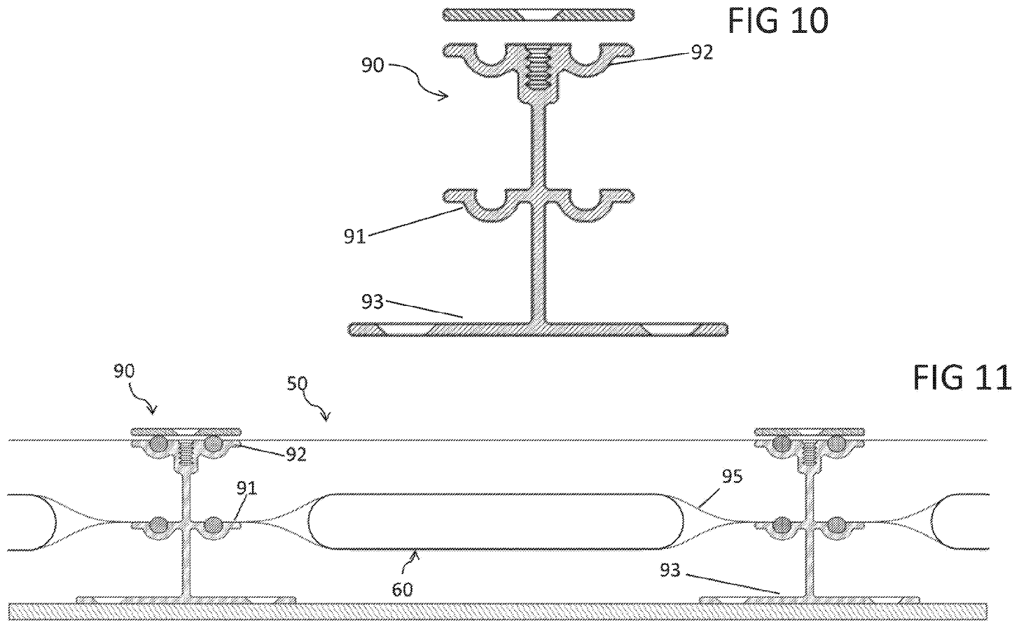

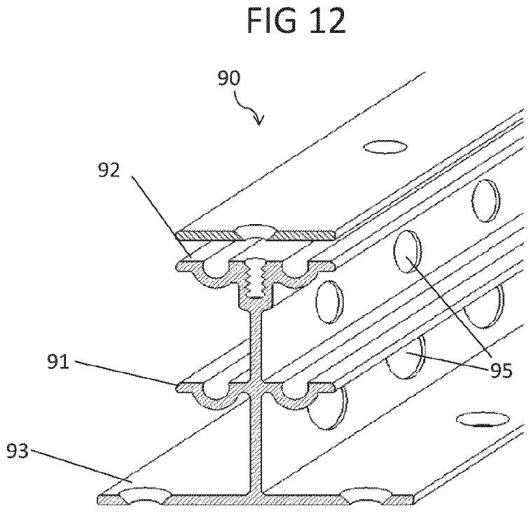

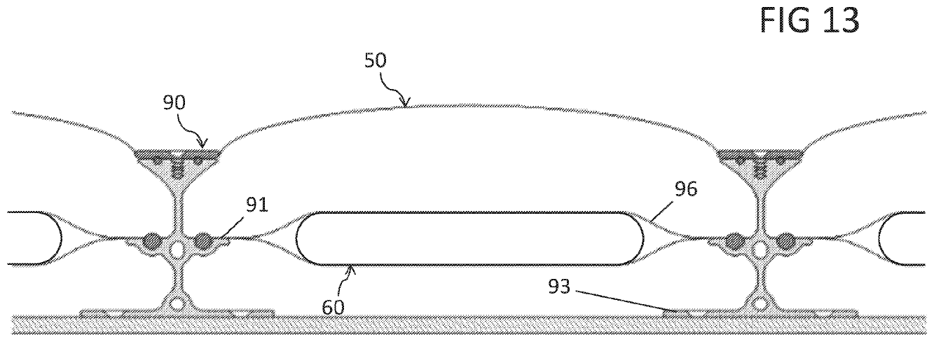

FIGS. 10 to 13 show that the chamber assembly may comprise a support structure (90) which may be comprised of a metal and/or plastic structure, for example an extruded structure, that extends linearly (following desired bioreactor array) on two sides, The structure may function as the structural support for the membrane bioreactor, in particular the upper and the bottom surfaces. The structure may comprise housing mechanisms or fittings (91, 92, 93) to fix and/or hold in place the bioreactors (91), the upper walls of the chamber (92) and the lower walls of the chamber (93). The ends on the modules can be closed by other support structure elements in order to create a closed chamber. The walls of the structure (see FIG. 12) may comprise holes (95) which enable gas to travel from one chamber section to another especially in embodiments which comprise an array of multiple chambers. The structures may hold the bioreactors directly or may be connected to further bioreactor support structures (96) such as cords or meshes which hold the bioreactors. FIGS. 11 and 13 show transverse cross sections across the bioreactors and chamber (see for example section B of FIG. 7a), and have multiple bioreactors positioned side-by-side, for example as seen in FIG. 3 or 4.

FIG. 13 shows an embodiment of the invention which is adapted to prevent the collection of water or other substances on horizontal surfaces of the apparatus, and so reduce light interference. In this drawing, the upper wall of the chamber has a rounded convex shape, so that water or other substances run off this surface. The upper wall can be rigid, and keep its convex shape by its own strength, or it can be flexible, and maintain its convex shape by inflation, that is, a higher pressure inside the chamber than externally. Another advantage of such embodiments is that condensation on the inside of the upper wall is encouraged to run away from positions directly above the bioreactor.

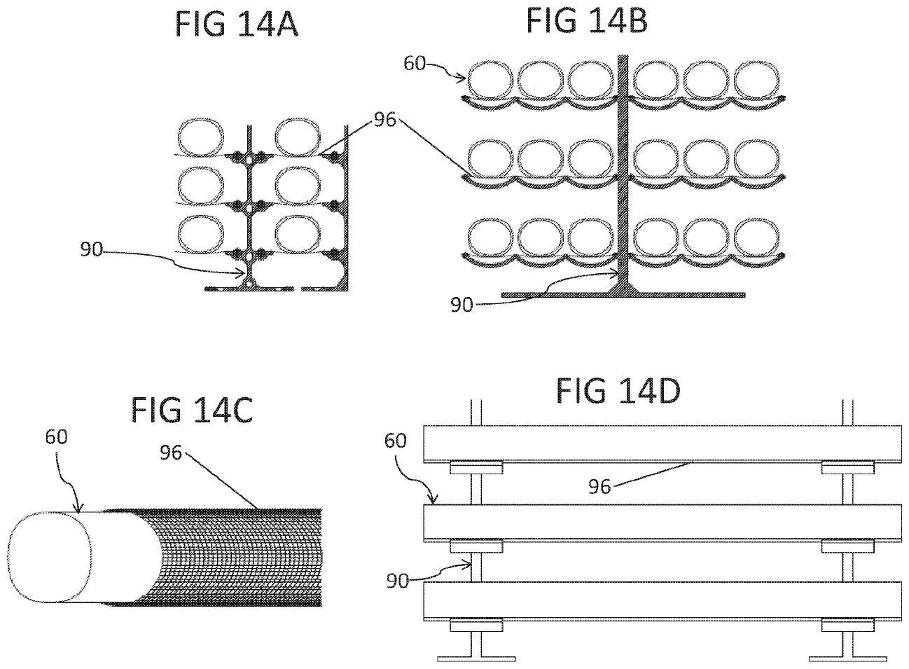

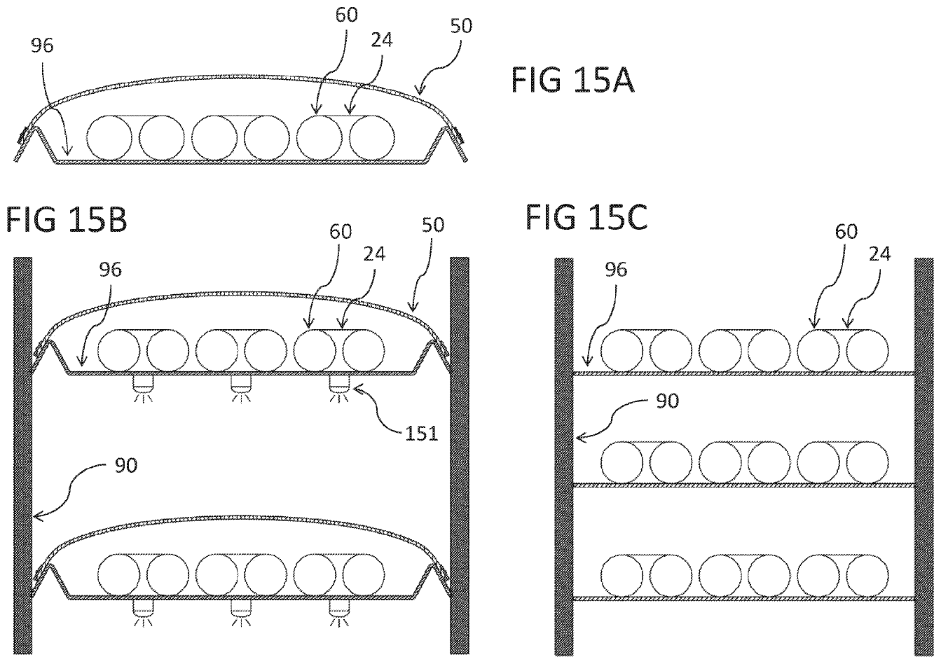

FIGS. 14a and 14b show an alternative example of support structures which may hold the bioreactors (60) in an array of shelves. In FIG. 14a, the three-dimensional array of bioreactors are suspended on a plurality of shelves comprising support structures (90) as shown, with bioreactor support structures (96) suspending the bioreactors themselves. FIG. 14b shows an alternative embodiment where an array of bioreactors are suspended by a support structure (90) comprising shelves made of a plurality of cradles, again with the bioreactors suspended by bioreactor support structures (96). FIG. 14c shows that the bioreactor support structure (96) can be a holding mesh (96), which may be perforated to allow gas to contact the bioreactors, and may surround substantially the whole circumference of the bioreactor. FIG. 14d shows a side view of a support structure (90) arranged as a plurality of shelves and supporting a plurality of bioreactors (60) on bioreactor support structures (96).

An exemplary configuration of the invention is as follows, suitable to grow Chlorella sp. in complete heterotrophic mode for the production of high protein content biomass. In a large warehouse with dimensions of approximately 250 m by 150 m, there are comprised numerous chambers comprising inflated tunnels constructed from a material that shields light in order to have a substantially dark environment inside the chamber. Each chamber is approximately 100 m long, 10 m wide and 3 m tall.

Inside each chamber is located a plurality of bioreactor arrays each comprising multiple tube-shaped bioreactors that define a flow circuit. Each tube array is installed on a shelf unit which supports the tubes on several vertical levels. Each shelf unit is approximately 70 cm wide, 2.5 m tall and 90 m long. A gap of approximately 70 cm between each shelf unit is left in order to enable maintenance and ventilation. Seven shelves are, arranged side by side in each chamber. Approximately 5 m of space is left between the outermost shelves and the chamber walls at each end, for ease of maintenance.

Each tube bioreactor compartment is approximately 30 mm in diameter, and is comprised of a polysiloxane membrane being 50 μm thick. Each bioreactor tube is approximately 5 m in length, and in each array, 18 bioreactors are connected in series with linear connectors, before a curved connector is used to connect a bioreactor to the subsequent bioreactor in an adjacent row. Each bioreactor array has 16 neighbouring rows of bioreactors. In addition, at the end of each row of bioreactors a connector is used to connect vertically to a bioreactor in an adjacent stack. 28 stacks are present in each bioreactor array. The arrangement and direction of flow through the rows and stacks of each bioreactor array is similar to that shown in FIG. 6A. Each bioreactor is surrounded by a mesh on all sides to provide support and maintain structural integrity. The cradles are further supported by fixing to the shelf units on which each tube sits, and also comprise a mesh structure to allow the gas of the chamber atmosphere to access the bioreactor membranes.

At one end of each chamber there is at least one air inlet connected to a filtering system and an impeller that directs outside atmospheric air into the chamber, with this inlet air being maintained at around 17° C. On the opposing end of the tunnel there is a purge (outlet) for the air. The impellers generate a positive pressure inside the chamber compared to the atmosphere surrounding the chambers, and thereby maintain inflation of the chamber tunnels. The chamber tunnels are also attached to the ceiling of the warehouse in any suitable manner to prevent collapse in case of impeller failure.

Bioreactor compartments are connected in series and separated by connector sections. Certain of the connectors comprise access ports to permit introduction of glucose and other nutrients where necessary, Connectors may also comprise static helicoid mixers. Vents to remove unwanted gas within the bioreactors themselves are located on the highest elevated point in the systems and suitably on the connectors linking bioreactors flowing in different directions.

An auxiliary system is installed and connected to the bioreactor array and comprises pumps to impart flow of the liquid media through the bioreactors, reservoirs for clean liquid media, and means for separating biomass from the liquid media, for inserting the initial inoculation of organisms to be cultured, for introducing cleaning fluids, for introducing sterilisation means, and for monitoring the status of the system.

Chlorella sp. is inoculated into the bioreactor system and grown to 10-15 g/l cell density. At the end of each growing period (typically every 12 to 24 hours) between 80 and 90% of the biomass in the system is harvested and the filtrate liquid is regenerated and recycled. The harvested biomass is taken into a biomass receptacle for further processing.

Related embodiments include an illumination system located between each shelf unit in order to deliver intermittent light and stimulate mixotrophic growth of mixotrophic microorganisms such as Chlorella sp. or Galdieria sp. Many eukaryotic microalgae are capable of mixotrophic growth and are able to grow fully photosynthetically or fully heterotrophically, or by using a combination of these methods. Chlorella sp. are notable examples.

In another embodiment the individual chambers are not included and instead the warehouse itself represents a single large chamber. Again, gas, typically atmospheric air, is introduced into this chamber; suitably after filtration by HEPA filters. This is particularly contemplated where the organism used are fully heterotrophic and light will not induce a phototrophic mode, or when the organism is an obligate mixotroph mode and the light present in the warehouse is sufficient to achieve growth. As such, windows may be provided to allow light to enter, and in some cases the chamber can be substantially fully transparent, such as a greenhouse.

### EXAMPLES

### Example 1

An experimental apparatus was constructed to demonstrate a system according to an embodiment of the present invention. In particular, the apparatus demonstrates that it can grow heterotrophic, chemoheterotrophic and/or mixotrophic organisms (which are contained in the liquid media inside a bioreactor of the type described herein) and that controlling the temperature of the gaseous atmosphere of a chamber containing the bioreactor of the type described herein results in the control of the temperature of a liquid or gel contained in the bioreactor. This further indicates that efficient O₂ and CO₂ gas transfer occurs through the membrane layer of the bioreactor to enable growth of heterotrophic, chemoheterotrophic and/or mixotrophic organisms in the liquid media contained by the bioreactor. Furthermore, it also indicates that the wall thickness of the membrane layer of the bioreactor enables efficient heat transfer through contact with the surrounding gaseous atmosphere.

The set-up is represented by a simplified schematic in FIG. 18. This set-up defines a system according to one embodiment of the present invention. With reference to FIG. 18 the majority of the features shown in this schematic are the same as those found in FIG. 9. In addition, there is shown: an outlet (143) to extract the liquid media from the apparatus (70) for its sampling and analysis or for the collection of the biomass; a series of elongated bioreactors according to the invention (60) as described herein in a shape of a tube and having end-reinforcement portions (144) in proximity to the ends of each bioreactor sections; conduits and connectors (24) that connect the bioreactor sections to each other and to the inlet (3) and outlet (4); a series of valves (140, 141, 142), a discharge outlet (145) and an auxiliary inlet (146) that are used during the cleaning, sterilization, start-up and inoculation procedures of the system. For example to replenish a dirty cleaning liquid previously used to clean the bioreactors with a new sterilising solution, the central valve (141) will be closed, the other two valves (140, 142) will be open and the pump (72) will continue to run to allow the dirty cleaning liquid to be discharged from the discharge outlet (145) and to allow the new sterilising solution to be introduced in the system from the auxiliary inlet (146). Another example is to replenish growth media consumed by the organisms and to remove liquid media from the system at the same time, the central valve (141) will be closed, the other two valves (140, 142) will be open and the pump (72) will continue to run to allow the liquid media to be discharged from the discharge outlet (145) and to allow the new liquid media with growth media to be introduced in the system from the auxiliary inlet (146).

The bioreactor was made of 12 membrane hose sections connected to each other in series as shown in FIG. 18. Each hose section was constructed from a single polysiloxane membrane layer, 200 μm thick, having permeability coefficient (ISO 15105-1) of oxygen (O₂) equal to approximately 400 Barrers, of carbon dioxide (CO₂) equal to approximately 2100 Barrers, of nitrogen (N₂) equal to approximately 200 Barrers, of hydrogen (H₂) equal to approximately 550 and of water vapour (H₂O) equal to approximately 30000 Barrers. Each hose section was constructed from a single membrane layer folded on and sealed to itself using a VVB adt-x silicone adhesive and heat pressed to create a continuous hose bioreactor section as shown by the cross section of the hose in FIG. 16B. Each membrane hose section was entirely enclosed by a fine transparent mesh to control the diameter of the hose to approximately 4.0 cm, and it was sitting on the flat bottom surface of the chamber (50).

The bioreactor was filled to its normal operating capacity with liquid media containing growth medium, glucose and Chlorella vulgaris (UTEX 259). Chlorella vulgaris is known to be a mixotroph that is able to use multiple trophic modes to grow: growth in the absence of light and the presence of an organic carbon source like glucose (in other words, growing chemoheterotrophically); or growth in the presence of light and CO₂, and the absence of an organic carbon source (in other words, growing photoautotrophically); or growth in other heterotrophic or phototrophic modes. For this specific case-study, Chlorella vulgaris was grown in complete darkness for all the duration of the experiment, and with the presence of glucose in the liquid media. The system is airtight, therefore gas exchange between the liquid media within the bioreactor and the atmosphere within the surrounding chamber occurs solely through the polysiloxane membrane layers of the bioreactor (60 ). Gas can be introduced or vented from the chamber via valves (7, 8) to control the pressure, humidity and gaseous mixture of the gaseous atmosphere in the chamber

The chamber (50) was constructed from a steel chassis (box) with an opening window on the superior surface glazed with a transparent ETFE layer approximately 200 μm thick. During the experiment, the opening window was entirely covered by an aluminium panel to make the inside of the chamber completely dark because the membrane hose sections were transparent. The chamber was designed to accommodate some sensors used for this case study:
1. Two Temperature sensors (PT100 from IFM),
2. A humidity sensor (LDH100 from IFM),
3. A pressure transmitter with ceramic measuring cell (IFM PA9028).

The reservoir (71) is designed to accommodate the sensors (75). The sensors (75) used for this case study were:
1. A pH sensor (“EASYFERM PLUS PHI ARC 120” from Hamilton),
2. A turbidity sensor (“DENCYTEE UNIT 120” from Hamilton),
3. A temperature sensor (IFM TM4431 PT100),
4. A pressure transmitter with ceramic measuring cell (IFM PA9026).

The liquid media temperature was maintained at 28° C. (with a variation kept within +−0.2° C. oscillation using PID control) by controlling the temperature of the gaseous atmosphere within the chamber. The air atmosphere within the chamber was heated to desired temperatures by an air heater device installed within the chamber that had to overcome the temperature of the air blown in the chamber (which was 21° C.) and the temperature of the surrounding air outside the chamber (which also was 21° C.). The liquid media was pumped throughout the system by a peristaltic pump “FMP50” from Boyser. One valve can divert the liquid media to an outlet (143) into a receptacle for biomass harvesting and further liquid media sampling when needed.

The experiment is divided in two runs:
During RUN 1 air is constantly blown through the inlet (7) in the chamber (50) and then out from the outlet (8).
During RUN 2 both chamber inlet (7) and outlet (8) are closed and the gaseous atmosphere within the chamber is sealed from other gases outside the chamber during the entire duration of the run.

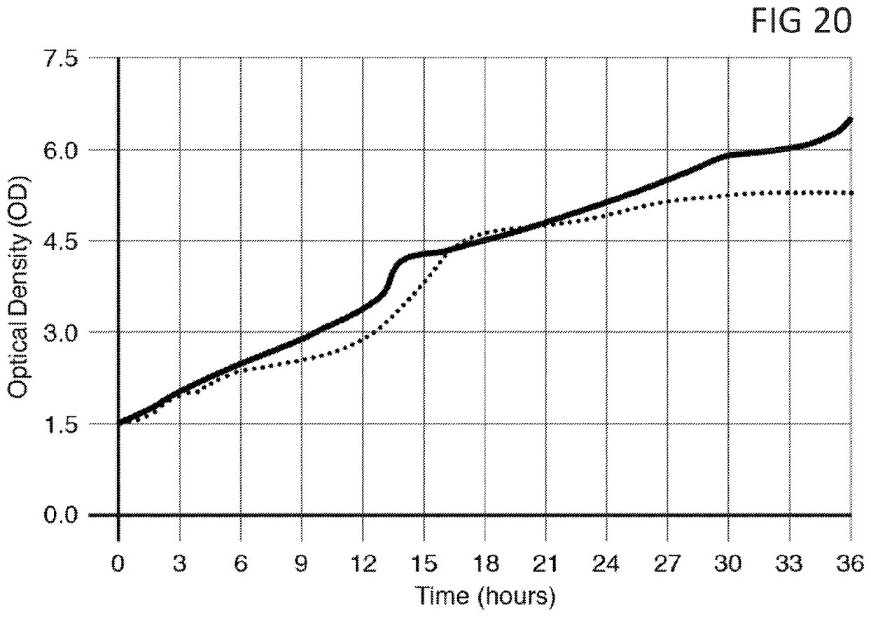

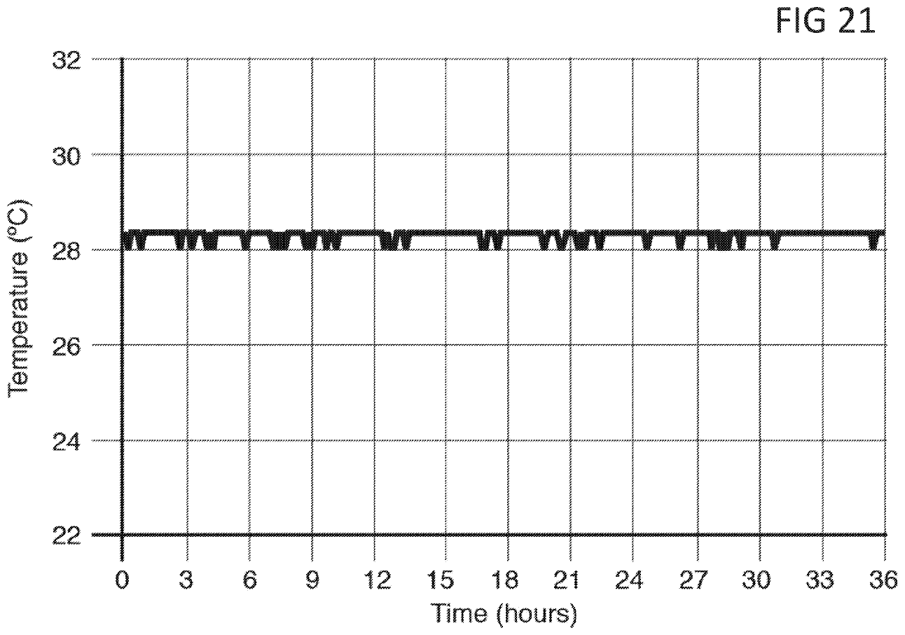

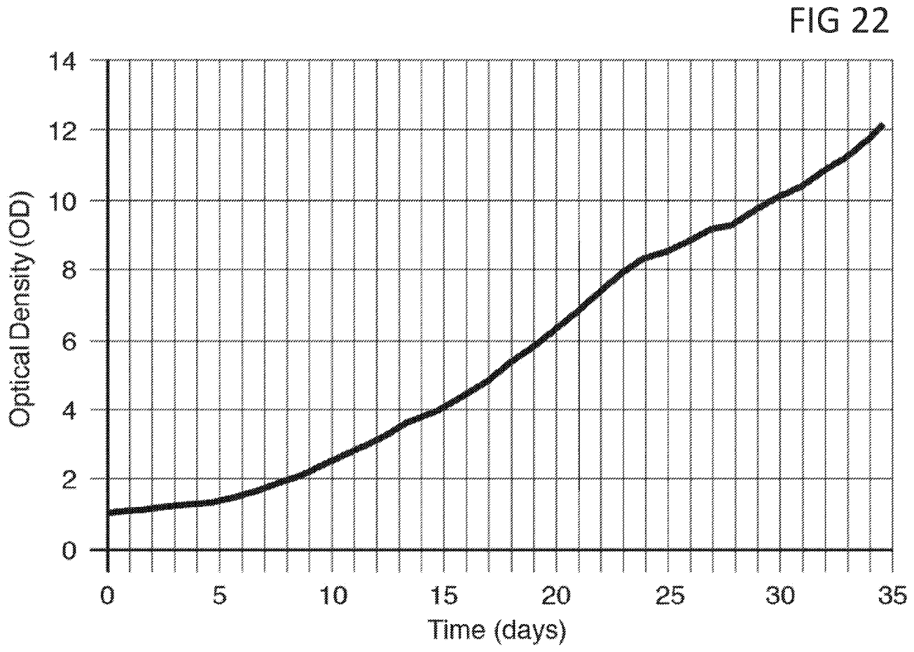

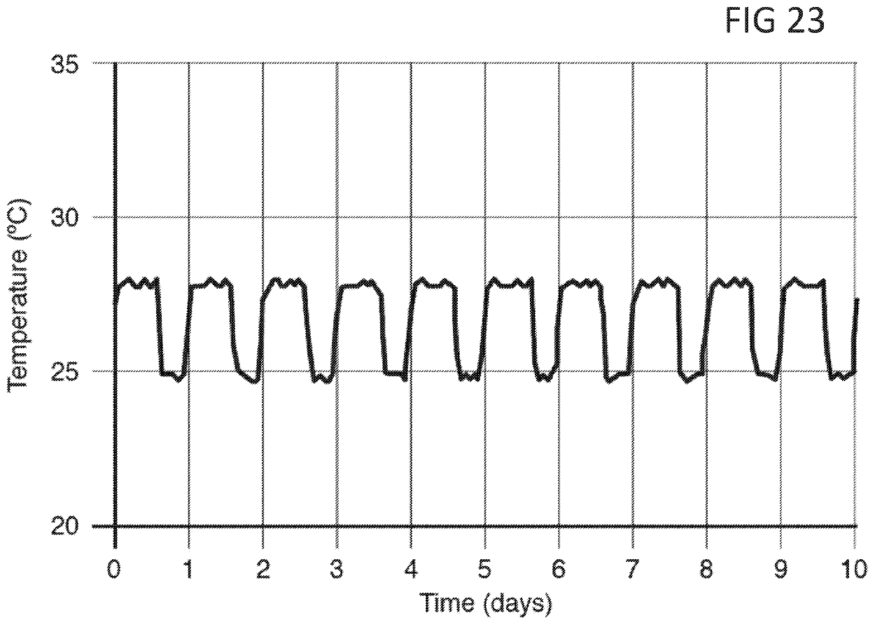

During RUN1, the optical density was seen to raise by approximately 4.8 OD in 36 hours and then to continue increasing after that; the optical density corresponds to the growth rate of the microorganism culture, and it is represented by the full line in the graph illustrated in FIG. 20. On the contrary, during RUN 2, the optical density decreased its increasing rate alter 18 hours, it ceased increasing after 31 hours, and it started decreasing after 35 hours (represented by the dotted line in the graph illustrated in FIG. 20). The lower growth rate experienced in RUN2 in respect to RUN1 is believed to be a consequence to the lower rate of oxygen exchange between the atmosphere in the chamber and the liquid media inside the bioreactor. During RUN2 the chamber was sealed to the outside air; therefore, no new air could replenish the oxygen concentration in the chamber that permeated through the membrane bioreactor into the liquid media and was consumed by the microorganisms.

This experiment shows that the technology works better when the level of oxygen in the chamber is controlled and maintained to desired concentration in order to maintain a constant osmotic gas flow between the atmosphere in the chamber and the liquid media in the membrane bioreactor. On the other hand, the experiment also shows that the technology underperforms when the chamber is sealed, which replicates a non-membrane bioreactor that is sealed to any outside gaseous atmosphere, in other words it replicates a non-gas-permeable bioreactor (like a non-gas-permeable tube or vessel bioreactor).

Furthermore, during the duration of both runs, the temperature in the liquid media was successfully maintained at desired conditions (between 28.0 and 28.2, using PID control) proving that the system can successfully control the liquid temperature by controlling the temperature of the gaseous atmosphere within the chamber. The liquid temperature during the duration of RUN1 is shown by the graph illustrated in FIG. 21.

Finally, this experiment shows that the technology is also effective with heterotrophic, chemoheterotrophic and/or mixotrophic organisms, that it can control the temperature and the concentration of certain gases, nutrients and metabolites in the liquid media by controlling the gaseous atmosphere in the chamber.

### Example 2

An experimental apparatus was constructed to demonstrate a system according to an embodiment of the present invention. In particular, the apparatus demonstrates that it can grow autotrophic and/or photoautotrophic organisms (which are contained in the liquid media inside a bioreactor of the type described herein) and that controlling the temperature of the gaseous atmosphere of a chamber containing the bioreactor (which in this particular case may also be termed a ‘photobioreactor’) of the type described herein results in the control of the temperature of a liquid or gel contained in the bioreactor. This further indicates that efficient CO₂ and O₂ gas transfer occurs through the membrane layer of the bioreactor, sufficient to enable the growth of autotrophic and/or photoautotrophic organisms in the liquid media contained by the bioreactor. Furthermore, it also indicates that the wall thickness of the membrane layer of the bioreactor enables efficient heat transfer through contact with the surrounding gaseous atmosphere.

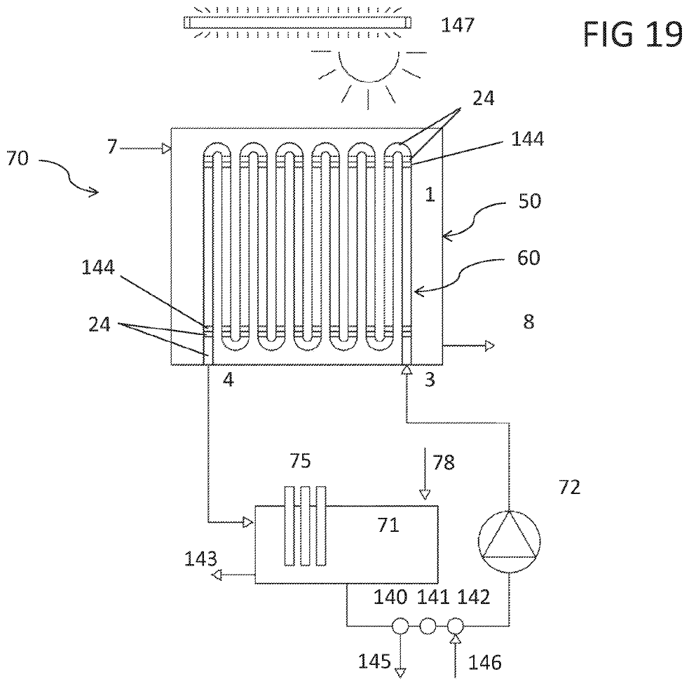

The case study set-up is represented by a simplified schematic in FIG. 19. This set-up defines a system according to one embodiment of the present invention. With reference to FIG. 19, the majority of the features shown in this schematic are the same as those found in FIG. 18. In addition, it is shown: a lighting source (147) that shine light onto the bioreactors.

With reference to this experimental apparatus, the majority of the features are the same as those of the experimental apparatus used in Example 1. The only differences were: an LED lighting device (VYPRx PLUS from Fluence) designed to emit specific wavelengths of electromagnetic radiation (light) corresponding to the needs of the microorganisms, and that was installed on top of the chamber's opening window; the aluminium panel installed on the opening window of the chamber (50) was removed to allow sufficient light through the window and to illuminate the transparent membrane hose bioreactor sections inside the chamber.

The bioreactor was filled to its normal operating capacity with liquid media containing growth medium and Arthrospira platensis, which is a microorganism known to be an obligate photoautotroph that can grow only in the presence of light and CO₂. For this specific case-study, Arthrospira platensis was grown on a 16 hours light and 8 hours dark cycle for most of the duration of the experiment, the light intensity was increased gradually from approximately a Photosynthetically Active Radiation (PAR) of 50 μmol·m²/s at the beginning of the experiment to approximately 300 μmol·m²/s towards the end of it. The liquid media didn't contain any organic carbon source. The system is airtight, therefore gas exchange between the liquid media within the bioreactor and the atmosphere within the surrounding chamber occurs solely through the polysiloxane membrane layers of the bioreactor (60). Gas can be introduced or vented from the chamber via valves (7, 8) to control the pressure, humidity and gaseous mixture of the gaseous atmosphere in the chamber.

The majority of the sensors utilised in this experiment are the same as those of the sensors used in Example 1, with the addition of one PAR sensor (LI-190R from Li-Cor) located on the top of the ETFE opening window of the chamber (50).

The liquid media temperature was maintained at 28° C. (with a variation kept within +−0.2° C. oscillation using PID control) during the light cycle and 25° C. (again with PID control maintaining a variation of +−0.2° C.) during the night cycle by controlling the temperature of the gaseous atmosphere within the chamber. The air atmosphere within the chamber was heated to desired temperatures by an air heater device installed within the chamber that had to overcome the temperature of the air blown in the chamber (which was 21° C.) intermittently to control the humidity, and the temperature of the surrounding air outside the chamber (which also was 21° C.). The humidity in the air chamber was also controlled in order to maintain 82% humidity or lower by pumping a gaseous mix with lower humidity. The liquid media was pumped throughout the system by a peristaltic pump “FMP50” from Boyser. One valve can divert the liquid media to an outlet (143) into a receptacle for biomass harvesting and further liquid media sampling when needed, while another valve (78) enables the insertion into the system of new growth medium from an auxiliary tank (71).

During the experiment, a gas mixture containing CO₂ was introduced in the chamber intermittently in order to enable enough osmotic flow of CO₂ through the membrane bioreactor into the liquid media to sustain the growth of the photoautotrophic microorganisms. The CO₂ concentration in the chamber was able to maintain the pH in the liquid media as desired (between 9.8-9.9 pH).

During the experiment, the optical density was seen to raise by approximately 11 OD in 35 days; the optical density corresponds to the growth rate of the microorganism culture inside the bioreactor, and it is represented by the full line in the graph illustrated in FIG. 20.

This experiment shows that the technology is also effective with autotrophic and/or photoautotrophic organisms and that it can control the temperature, pH and the concentration of gases, nutrients and metabolites in the liquid media by controlling the gaseous atmosphere in the chamber. Furthermore, during the duration of both runs, the temperature in the liquid media was successfully maintained at desired conditions (approximately 28.0+−0.2 during the light cycle and 25.0+−0.2 during the dark cycle) proving that the system can successfully control the liquid temperature by controlling the temperature of the gaseous atmosphere within the chamber. The liquid temperature during 10 days of the experiment is shown by the graph illustrated in FIG. 23.

These two experiments (described in Examples 1 and 2) prove that the technology works for phototrophs, chemotrophs and mixotrophs.

Although particular embodiments of the invention have been disclosed herein in detail, this has been done by way of example and for the purposes of illustration only. The aforementioned embodiments are not intended to be limiting with respect to the scope of the appended claims, which follow. It is contemplated by the inventors that various substitutions, alterations, and modifications may be made to the invention without departing from the spirit and scope of the invention as defined by the claims

1. An apparatus for the production of biomass or a bioproduct, the apparatus comprising:
(i) at least one elongate bioreactor, the bioreactor comprised of at least one outer membrane layer that defines a substantially tubular compartment that is capable of being filled with a liquid or gel, wherein the membrane layer is comprised of a material that is permeable to gas transfer across the membrane layer;
(ii) a chamber comprising walls that define and enclose a gaseous atmosphere within, wherein at least a part of the bioreactor is located inside the chamber; and
(iii) a control system which controls the composition of the atmosphere within the chamber, wherein gas transfer occurs across the membrane layer of the bioreactor between the tubular compartment and the atmosphere comprised within the chamber.

2. The apparatus of claim 1, wherein chamber is in the form of a tank, a vessel, a barrel, a tent, a warehouse, an inflated structure, or a room.

3. The apparatus of claim 1, wherein the atmosphere within the chamber may be elevated to a pressure greater than or less than atmospheric pressure.

4. The apparatus of claim 1, wherein the control system is configured to alter the atmospheric composition of the chamber by:
(i) introducing an O₂-containing gas
(ii) depleting CO₂ concentration; and/or
(iii) introducing steam.

5. The apparatus of claim 1, wherein the chamber further comprises:
(i) a sterilisation system;
(ii) gas circulatory apparatus; and/or
(iii) a source of illumination, optionally wherein the source of illumination emits visible and/or UV light.

6. The apparatus of claim 1, wherein at least one or a part of one wall of the chamber permits the transmission therethrough of visible light into the interior of the chamber.

7. The apparatus of claim 1, wherein the chamber comprises an assembly for supporting the at least one elongate bioreactor within preferably wherein the assembly comprises a plurality of armatures arranged in either a horizontal or vertical parallel or anti-parallel array.

8. The apparatus of claim 7, wherein the assembly comprises at least one cradle configured to support the at least one elongate bioreactor.

9. The apparatus of claim 8, wherein the cradle substantially encloses all or a part of the elongate bioreactor, preferably wherein the cradle is comprised of a mesh or a perforated sheet material, such that atmospheric circulation may be permitted via the perforations of the sheet material.

10. The apparatus of claim 1, wherein the elongate bioreactor is comprised of one or more hose sections, wherein each hose section is comprised of a gas permeable polymer membrane.

11. The apparatus of claim 10, wherein the gas permeable polymer membrane is selected from: silicones, polysiloxanes, polydimethylsiloxanes (PDMS), fluorosilicone, organosilicones, cellulose (including plant cellulose and bacterial cellulose), cellulose acetate (celluloid), nitrocellulose, and cellulose esters.

12. The apparatus of claim 11, wherein the membrane has:
(i) an oxygen permeability of at least 350, at least 400, at least 450, at least 550, at least 650, at least 750, suitably at least 820 Barrers;
(ii) a carbon dioxide permeability of at least 2000, at least 2500, at least 2600, at least 2700, at least 2800, at least 2900, at least 3000, at least 3100, at least 3200, at least 3300, at least 3400, at least 3500, at least 3600, at least 3700, at least 3800, suitably at least 3820 Barrers; and/or
(iii) a water vapour permeability of at least 5000, at least 10000 Barrer, at least 15000 Barrer, at least 20000 Barrer, at least 25000 Barrer, at least 30000 Barrer, at least 35000 Barrer, at least 40000, at least 60000 and typically at least 80000 Barrer.

13. The apparatus of claim 1, wherein the membrane has a thickness of at least 10 μm and at most 1 mm, suitably at least 20 μm and at most 500 μm, optionally at least 20 μm and at most 200 μm.

14. The apparatus of claim 10, wherein the one or more hose sections are joined by one or more connectors that facilitate fluid communication between the one or more hose sections.

15. The apparatus of claim 14, wherein the one or more connectors comprise a valve which is operable to reduce or stop fluid communication between the one or more hose sections.

16. The apparatus of claim 1, wherein the bioreactor is in fluid communication with an auxiliary system.

17. The apparatus of claim 1, wherein the one or more bioreactors comprise a liquid cellular growth medium.

18. The apparatus of claim 17, wherein the one or more bioreactors comprise a microbial or algal organism selected from: a photoautotroph, a chemotroph and a mixotroph.

19. The apparatus of claim 18, wherein the organism is selected from one or more of Cyanobacteria; Protobacteria; Spirochaetes; Gram Positive bacteria; green filamentous bacteria; such as Chloroflexia; Planctomycetes; Bacteroides cytophaga; Thermotoga; Aquifex; halophiles; Methanosarcina; Methanobacterium; Methanococcus; Thermococcus celer; Thermoproteus; Pyrodictium; Entamoebae; slime moulds; such as Mycetozoa; Ciliates; Trichomonads; Microsporidia; Diplomonads; Excavata; Amoebozoa; Choanoflagellates; Rhizaria; Foraminifera; Radiolaria; Diatoms; Stramenopiles; brown algae; red algae; green algae; snow algae; Haptophyta; Cryptophyta; Alveolata; Glaucophytes; phytoplankton; plankton; Percolozoa; Rotifera; and cells or whole organisms from animals, fungi, or plants.

20. The apparatus of claim 17, wherein the bioreactor comprises a eukaryotic cell culture; suitably an animal, or plant cell culture; optionally a mammalian cell culture.

21. The apparatus of claim 18, wherein the bioreactor comprises a human cell culture.

22. The apparatus of claim 1, wherein the control system is further configured to control the temperature of the atmosphere within the chamber.

23. A method for manufacturing biomass, the method comprising:
(i) providing an apparatus comprising:
(a) at least one elongate bioreactor, the bioreactor comprised of at least one outer membrane layer that defines a substantially tubular compartment that is capable of being filled with a liquid or gel, wherein the membrane layer is comprised of a material that is permeable to gas transfer across the membrane layer;
(b) a chamber comprising walls that define and enclose a gaseous atmosphere within wherein at least a part of the at least one bioreactor is located inside the chamber;
(c) a control system which controls the composition of the atmosphere within the chamber;
the at least one elongate bioreactor comprising a liquid cellular growth medium and a microbial or algal organism selected from a chemoheterotroph and a mixotroph, and/or a eukaryotic cell culture;
(ii) culturing the organisms or cell cultures within the one or more bioreactors; and
(iii) separating at least a part of the biomass present within the liquid media.
